(19) United States
(12) Patent Application Publication
(10) Pub. No.: US 2008/0306115 A1
(43) Pub. Date: Dec. 11, 2008

(54) Midazole-4-Carboxamide Derivatives For Use As Cb1 Modulators

(76) Inventor: Leifeng Cheng, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 12/066,949

(22) PCT Filed: Sep. 12, 2006
(86) PCT No.: PCT/GB2006/003358

§ 371 Date: Aug. 4, 2008

(30) Foreign Application Priority Data
Sep. 15, 2005 (GB) 0518819.8

(51) Int. Cl. A61K 31/4164; A61P 3/04; A61P 25/00; A61P 11/00; A61K 31/454; A61K 31/4439; A61P 1/00; C07D 233/90

(52) U.S. Cl. 514/326; 548/333.5; 514/400; 514/341

(57) ABSTRACT

The present invention relates to substituted 1,2-diphenylimidazol-4-carboxamide compounds of formula I

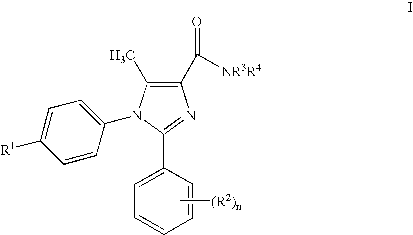

to processes for preparing such compounds, their use in the treatment of obesity, psychiatric and neurological disorders, to methods for their therapeutic use and to pharmaceutical compositions containing them.

### FIELD OF INVENTION

The present invention relates to certain 1,2-diarylimidazole-4-carboxamide compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354).

WO04/60367 and WO2004/099130 disclose that certain diaryl imidazoles and triazoles are useful as COX-1 inhibitors useful in the treatment of inflammation. Compounds exemplified in these applications are disclaimed from the claims of the present invention. DD 140966 discloses that certain imidazolecarboxylic acid anilides are useful as plant growth regulators. Compounds exemplified in this application are disclaimed from the claims of the present invention.

WO 03/007887 and WO03/075660 disclose certain 4,5-diarylimidazole-2-carboxamides as CB₁ modulators.

WO03/27076 and WO 03/63781 disclose certain 1,2-diarylimidazole-4-carboxamides which are CB₁ modulators. Compounds exemplified in these applications are disclaimed from the claims of the present invention.

WO03/40107 discloses certain 1,2-diarylimidazole-4-carboxamides as being useful in the treatment of obesity and obesity-related disorders.

However, there is a need for CB₁ modulators with improved physicochemical properties and/or DMPK properties and/or pharmacodynamic properties.

Co-pending application number PCT/GB2005/001153 discloses CB1 antagonists of formula (A)

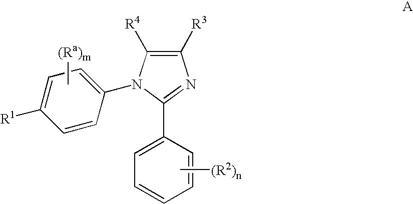

and pharmaceutically acceptable salts thereof, in which
R¹ represents a) a C₁₋₆alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R¹ represents a C₁₋₁₀alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;
R¹ represents halo, a C₁₋₃alkyl group or a C₁₋₃alkoxy group;
m is 0, 1, 2 or 3;
R¹ represents a C₁₋₃alkyl group, a C₁₋₃alkoxy group, hydroxy, nitro, cyano or halo
n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl) C₁₋₃alkylene group optionally substituted by 1, 2, or 3 groups represented by W;
a group—(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl; a group—(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃a koxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl;
with the proviso that when n is 1 then R² is not methoxy in either the 2-position or the 4-position of the phenyl ring and the further proviso that R¹ is not methylsulfonylamlino, methoxy or CF₃O—. Compounds exemplified in this application are disclaimed from the present application.

However, there is a need for CB₁ modulators with improved physicochemical properties and/or DMPK properties and/or pharmnacodynamic properties. A select group of compounds has been found that addresses this need.

### DESCRIPTION OF THE INVENTION

The invention relates to a compound of formula (I)

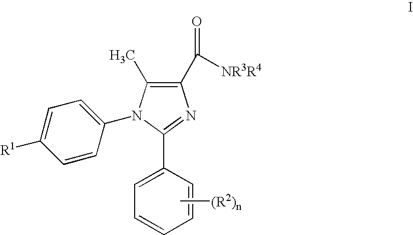

wherein R¹ represents a group R⁵O— in which R⁵ represents a C₃₋₇alkyl group substituted by one or more fluoro or R⁵ represents a C₃₋₇alkylsulphonyl group which is substituted by one or more fluoro;
R² represents a C₁₋₄alkyl group, fluoro, chloro or cyano wherein each R² is independently selected when n is >1;
R³ represents H; and
R⁴ represents a) cyclohexyl optionally substituted by one or more of the following: hydroxy, fluoro, amino, mono or diC₁₋₃alkylamino b) piperidino substituted by one or more hydroxy c) unsubstituted piperidino but only when one of the following applies: R¹ represents 3-fluoropropylsulphonyloxy, or 3,3,3-trifluoropropoxy or 3-fluoropropoxy or at least one R² represents methyl; d) phenyl substituted by one or more trifluoromethoxy e) pyridyl substituted by one or more of the following: a C₁₋₄allyl group; trifluoromethyl; or fluoro; provided that R⁴ is not 5-trifluoromethyl-2-pyridyl or f) a C₄₋₉alkyl group optionally substituted by one or more hydroxy;
n is 1, 2 or 3
and pharmaceutically acceptable salts thereof.

Further values of R¹, R², R⁴ and n in compounds of formula I now follow. It will be understood that such values may be used where appropriate with any of the definitions, claims or embodiments defined hereinbefore or hereinafter.

In a first group of compounds of formula I, R¹ represents a (C₃-C₄ alkyl) sulphonyloxy group substituted by one or more fluoro. Alternatively, R¹ represents (C₃-C₄ alkoxy) group substituted by one or more fluoro. In each case particularly the fluoro is in the terminal position of the alkyl chain. Particularly R¹ represents one or more of the following: 4,4,4-trifluorobutyl-1-sulfonyloxy, 3,3,3-trifluoropropyl-1-sulfonyloxy, 3-fluoropropyl-1-sulfonyloxy, 3,3,3-trifluoropropoxy or 3-fluoropropoxy.

In a second group of compounds of formula I, R² represents chloro, fluoro, cyano or methyl and n is 1 or 2 for example: 3-cyano-5-fluoro; 3-cyano; 2,4-dichloro; 2-chloro; or 4-chloro-2-methyl.

In a third group of compounds of formula I, R⁴ represents cyclohexyl substituted by one or more of the following: hydroxy, fluoro, amino, mono or di C₁₋₃alkylamino; for example 2-hydroxycyclohexyl, 3-hydroxycyclohexyl, 2-aminocyclohexyl, 3-aminocyclohexyl, 2-dimethylaminocyclohexyl, 3-dimethylaminocyclohexyl or 4,4-difluorocyclohexyl. In one group of compounds the substituent is in the 2 or 3 position. In another group of compounds the substituent on the cyclohexyl ring is in the cis conformation with respect to the nitrogen of the amide. In another group of compounds the substituent on the cyclohexyl ring is in the trans conformation with respect to the nitrogen of the amide.

In a fourth group of compounds of formula I, R⁴ represents piperidino substituted by one or more hydroxy, for example 3-hydroxypiperidino or 4-hydroxypiperidino.

In a fifth group of compounds of formula I, R⁴ represents unsubstituted piperidino but only when one of the following applies: R¹ represents 3-fluoropropylsulphonyloxy or 3,3,3-trifluoropropoxy or 3-fluoropropoxy or at least one R² represents methyl.

In a sixth group of compounds of formula I, R⁴ represents phenyl substituted by one or more trifluoromethoxy, for example 4-trifluoromethoxyphenyl.

In a seventh group of compounds of formula I, R⁴ represents pyridyl substituted by one or more of the following: a C₁₋₄alkyl group; trifluoromethyl; or fluoro; provided that R⁴ is not 5-trifluoromethyl-2-pyridyl, for example methylpyridyl e.g. 5-methyl-2-pyridyl, or for example fluoropyridyl e.g. 6-fluoro-2-pyridyl, or trifluoromethylpyridyl for example 6-trifluoromethyl-3-pyridyl.

In an eighth group of compounds of formula I, R⁴ represents a C₄₋₇alkyl group optionally substituted by one or more hydroxy; for example 1-ethylbutyl or 1-hydroxymethyl-3-methylbutyl.

In a ninth group of compounds of formula I, R⁴ represents a C₄₋₇alkyl group substituted by one or more hydroxy; for example 1-hydroxymethyl-3-methylbutyl.

In a tenth group of compounds of formula I, R⁴ represents cyclohexyl. “Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

Specific compounds of the invention are one or more of the following:
4-[2-(2,4-dichlorophenyl)-5-methyl-4-({[6-(trifluoromethyl)pyridin-3-yl]amino}carbonyl)-1H-imidazol-1-yl]phenyl 3,3,3-trifluoropropane-1-sulfonate;
4-(2-(2,4-dichlorophenyl)-5-methyl-4-{[(5-methylpyridin-2-yl)amino]carbonyl}-1H-imidazol-1-yl)phenyl 3,3,3-trifluoropropane-1-sulfonate;
4-(2-(2,4-dichlorophenyl)-4-{[(6-fluoropyridin-3-yl)amino]carbonyl}-5-methyl-1H-imidazol-1-yl)phenyl 3,3,3-trifluoropropane-1-sulfonate;
4-[2-(2,4-dichlorophenyl)-4-({[(1S,2R)-2-hydroxycyclohexyl]amino}carbonyl)-5-methyl-1H-imidazol-1-yl]phenyl 3,3,3-trifluoropropane-1-sulfonate;
and 4-[2-(2,4-dichlorophenyl)-4-({[(1R,2S)-2-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[(1S,2S)-2-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[(1R,2R)-2-hydroxycyclohexyl]amino}carbonyl)-5-methyl-1H-imidazol-1-yl]phenyl 3,3,3-trifluoropropane-1-sulfonate;
4-{2-(2,4-dichlorophenyl)-5-methyl-4-[(piperidin-1-ylamino)carbonyl]-1H-imidazol-1-yl}phenyl 3-fluoropropane-1-sulfonate;
4-[2-(2,4-dichlorophenyl)-5-methyl-4-({[4-(trifluoromethoxy)phenyl]amino}carbonyl)-1H-imidazol-1-yl]phenyl 3,3,3-trifluoropropane-1-sulfonate;
3,3,3-trifluoropropane-1-sulfonic acid 4-[2-(2,4-dichlorophenyl)-4-(3-hydroxypiperidin-1-ylcarbamoyl)-5-methylimidazol-1-yl]phenyl ester;
3,3,3-trifluoropropane-1-sulfonic acid 4-[2-(2,4-dichlorophenyl)-4-(4-hydroxypiperidin-1-ylcarbamoyl)-5-methyl-imidazol-1-yl]phenyl ester;
(−)4-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate;
2-(2,4-dichlorophenyl)-5-methyl-1-[4-(3,3,3-trifluoropropoxy)phenyl]-1H-imidazole-4-carboxylic acid (2-hydroxycyclohexyl)amide;
(+)4-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate;
(+)4-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate;
(−)4-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate;
4-[4-({[(1S,2R)-2-aminocyclohexyl]amino}carbonyl)-2-(2,4-dichlorophenyl)-5-methyl-1H-imidazol-1-yl]phenyl 3,3,3-trifluoropropane-1-sulfonate;
4-[2-(2,4-dichlorophenyl)-4-({[(1S,2R)-2-(dimethylamino)cyclohexyl]amino}carbonyl)-5-methyl-1H-imidazol-1-yl]phenyl 3,3,3-trifluoropropane-1-sulfonate;
2-(2,4-dichlorophenyl)-5-methyl-N-piperidin-1-yl-1-[4-(3,3,3-trifluoropropoxy)phenyl]-1H-imidazole-4-carboxamide;
N-cyclohexyl-2-(2,4-dichlorophenyl)-5-methyl-1-[4-(3,3,3-trifluoropropoxy)phenyl]-1H-imidazole-4-carboxamide;
2-(2,4-dichlorophenyl)-N-(4,4-difluorocyclohexyl)-5-methyl-1-[4-(3,3,3-trifluoropropoxy)phenyl]-1H-imidazole-4-carboxamide;
2-(2,4-dichlorophenyl)-5-methyl-N-(5-methylpyridin-2-yl)-1-[4-(3,3,3-trifluoropropoxy)phenyl]-1H-imidazole-4-carboxamide;
2-(2,4-dichlorophenyl)-1-[4-(3-fluoropropoxy)phenyl]-5-methyl-N-piperidin-1-yl-1H-imidazole-4-carboxamide;
N-cyclohexyl-2-(2,4-dichlorophenyl)-1-[4-(3-fluoropropoxy)phenyl]-5-methyl-1H-imidazole-4-carboxamide;
2-(2,4-dichlorophenyl)-1-[4-(3-fluoropropoxy)phenyl]-N-[(cis)-2-hydroxycyclohexyl]-5-methyl-1H-imidazole-4-carboxamide;
2-(2,4-dichlorophenyl)-N-(4,4-difluorocyclohexyl)-1-[4-(3-fluoropropoxy)phenyl]-5-methyl-1H-imidazole-4-carboxamide;
2-(2,4-dichlorophenyl)-1-[4-(3-fluoropropoxy)phenyl]-5-methyl-N-(5-methylpyridin-2-yl)-1H-imidazole-4-carboxamide;
4-[2-(2,4-dichlorophenyl)-4-({[cis-3-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[trans-3-hydroxycyclohexyl]amino}carbonyl)-5-methyl-1H-imidazol-1-yl]phenyl 3,3,3-trifluoropropane-1-sulfonate;
4-{2-(2-chlorophenyl)-4-[(cyclohexylamino)carbonyl]-5-methyl-1H-imidazol-1-yl}phenyl 3,3,3-trifluoropropane-1-sulfonate;
4-[2-(2-chlorophenyl)-4-({[cis-2-hydroxycyclohexyl]amino}carbonyl)-5-methyl-1H-imidazol-1-yl]phenyl 3,3,3-trifluoropropane-1-sulfonate;
2-(2,4-dichlorophenyl)-1-[4-(3-fluoropropoxy)phenyl]-5-methyl-N-piperidin-1-yl-1H-imidazole-4-carboxamide;
2-(2,4-dichlorophenyl)-N-[(cis)-2-hydroxycyclohexyl]-5-methyl-1-[4-(3,3,3-trifluoropropoxy)phenyl]-1H-imidazole-4-carboxamide;
4-{2-(2,4-dichlorophenyl)-4-[(4-hydroxycyclohexyl)carbamoyl]-5-methyl-1H-imidazol-1-yl}phenyl 3,3,3-trifluoropropane-1-sulfonate;
3,3,3-trifluoropropane-1-sulfonic acid 4-[2-(3-cyano-5-fluorophenyl)-4-(4,4-difluorocyclohexylcarbamoyl)-5-methylimidazol-1-yl]phenyl ester;
3,3,3-trifluoropropane-1-sulfonic acid 4-[2-(3-cyanophenyl)-4-(1-hydroxymethyl-3-methylbutylcarbamoyl)-5-methylimidazol-1-yl]phenyl ester;
3,3,3-trifluoropropane-1-sulfonic acid 4-[4-((2-aminocyclohexylcarbamoyl)-2-(3-cyano-5-fluorophenyl)-5-methylimidazol-1-yl]phenyl ester;
3,3,3-trifluoropropane-1-sulfonic acid 4-[2-(3-cyano-5-fluorophenyl)-4-(3-dimethylaminocyclohexylcarbamoyl)-5-methylimidazol-1-yl]phenyl ester;
3,3,3-trifluoropropane-1-sulfonic acid 4-[2-(3-cyano-5-fluorophenyl)-4-((1S,2R)-2-hydroxycyclohexylcarbamoyl)-5-methylimidazol-1-yl]phenyl ester;
3,3,3-trifluoropropane-1-sulfonic acid 4-[2-(3-cyanophenyl)-4-(2-hydroxy-cyclohexylcarbamoyl)-5-methylimidazol-1-yl]phenyl ester;
3,3,3-trifluoropropane-1-sulfonic acid 4-[2-(3-cyano-5-fluorophenyl)-4-(3-hydroxycyclohexylcarbamoyl)-5-methylimidazol-1-yl]phenyl ester;
3,3,3-trifluoropropane-1-sulfonic acid 4-[2-(2-chlorophenyl)-4-(2-hydroxy-cyclohexylcarbamoyl)-5-methylimidazol-1-yl]phenyl ester;
3,3,3-trifluoropropane-1-sulfonic acid 4-[2-(2-chlorophenyl)-4-(4,4-difluoro-cyclohexylcarbamoyl)-5-methylimidazol-1-yl]phenyl ester;
3,3,3-trifluoropropane-1-sulfonic acid 4-[2-(4-chloro-2-methylphenyl)-5-methyl-4-(piperidin-1-ylcarbamoyl)imidazol-1-yl]phenyl ester;
2-(2,4-dichlorophenyl)-5-methyl-1-[4-(3,3,3-trifluoropropoxy)phenyl]-1H-imidazole-4-carboxylic acid (3-hydroxycyclohexyl)amide;
3-Fluoropropane-1-sulfonic acid 4-[2-(2,4-dichlorophenyl)-4-((1S,2R)-2-hydroxy-cyclohexylcarbamoyl)-5-methylimidazol-1-yl]phenyl ester
4,4,4-trifluorobutane-1-sulfonic acid 4-[2-(3-cyano-5-fluorophenyl)-4-(1-ethyl-butylcarbamoyl)-5-methylimidazol-1-yl]phenyl ester;
   and pharmaceutically acceptable salts thereof.

### Methods of Preparation

The compounds of the invention may be prepared as outlined below according to any of the following methods. However, the invention is not limited to these methods, the compounds may also be prepared as described for structurally related compounds in the prior art.

Compounds of formula I in which R¹ represents a group R⁵ O may be prepared by reacting a compound of formula II

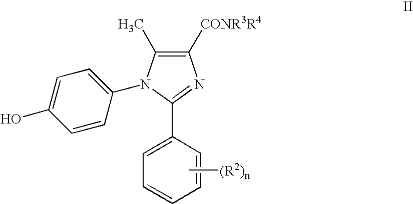

in which R², R³, R⁴ and n are as previously defined with a group R^{1A}-X in which R^{1A} represents a group such that R^{1A}O represents R¹ and X represents a leaving group for example halo, at a temperature in the range of −25 to 150° C., in the presence of an inert solvent, for example dichloromethane, and optionally in the presence of a base for example triethylamine or pyridine.

Compounds of formula I in which R¹, R², R³, R⁴ and n are as previously defined may be prepared by reacting a compound of formula III

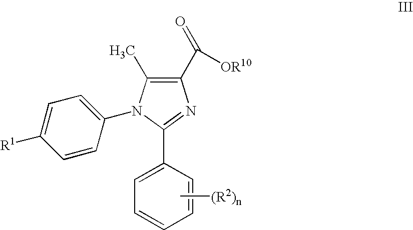

in which R¹, R² and n are as previously defined and R¹⁰ represents H or a C₁₋₆alkyl group with a compound of formula IV or a salt thereof

R³R⁴NH₂ IV

in which R³ and R⁴ are as previously defined, in an inert solvent, for example toluene, in the presence of a Lewis Acid, for example trimethylaluminium, at a temperature in the range of −25° C. to 150° C. when R¹⁰ is a C₁₋₆alkyl group; or alternatively when R¹⁰ is H by reacting a compound of formula III with a chlorinating agent for example oxalyl chloride, and then reacting the acid chloride produced with an amine of formula IV in an inert solvent, for example dichloromethane, in the presence of a base, for example triethylamine or pyridine, at a temperature in the range of −25° C. to 150° C. Compounds of formula I may also be prepared by deprotecting a compound of formula IA in which one of the substituents on R⁴ is in a protected form to give a compound of formula I. For example a compound of formula I in which R⁴ represents aminocyclohexyl may be prepared from a compound of formula IA in which R⁴ represents a protected-amino cyclohexyl group, for example tert-butoxycarbonylaminocyclohexyl, by deprotection methods known to those skilled in the art for example by reaction with thionyl chloride in methanol.

Certain intermediate compounds are believed to be novel and form part of the present invention, particularly compounds of formula III as defined above and including each and every definition of R¹ given previously.

Compounds of formula II and III may be prepared as shown in the examples and adaptations thereof or by analogous methods known to those skilled in the art. It will be appreciated by those skilled in the art that during the reaction sequence certain functional groups will require protection followed by deprotection at an appropriate stage (see “Protective Groups in Organic Synthesis”, 3rd Edition (1999) by Greene and Wuts).

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, and cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, and cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barrésyndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of is total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

The compounds of the present invention may also be suitable for use in the regulation of bone mass and bone loss and therefore useful in the treatment of osteoporosis and other bone diseases.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid; another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof. According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier. According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof. According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man. According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB 1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 2000 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x) ÙD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar. For example the IC50 of Example 3 is 1.2 nM

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example increasing the free fraction of drug) or solubility compared to representative reference CB1 antagonists/inverse agonist agents. The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES

### Abbreviations

ACN acetonitrile
(Boc)₂O di-tert-butyl-dicarbonate
DCM dichloromethane
DEAD diethyl azodicarboxylate
DMF dimethylformamide
DEA diethylamine
EtOAc ethyl acetate
MeOH methanol
rt room temperature
TBAF tetrabutylammonium fluoride
TBDMSCl: tert-butyldimethylsilyl chloride
TEA triethylamine
THF tetrahydrofuran
TLC thin layer chromatography
t triplet
singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC with a mass triggered fraction collector, Shimadzu QP 8000 single quadrupole mass spectrometer equipped with 19×100 mm C8 column. The mobile phase used was, if nothing else is stated, acetonitrile and buffer (0.1 M NH₄Ac:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

### Examples of the Invention

### Example 1

### 4-[2-(2,4-dichlorophenyl)-5-methyl-4-({[6-(trifluoromethyl)pyridin-3-yl]amino}carbonyl)-1H-imidazol-1-yl]phenyl 3,3,3-trifluoropropane-1-sulfonate

### Step 1

### N-(4-Benzyloxyphenyl)-2,4-dichloro-benzamidine

4-Benzyloxyaniline hydrochloride (5.0 g, 21.2 mmol) was added dropwise to a solution of ethylmagnesium bromide (44.5 ml, 1M in THF, 44.5 mmol) in 25 ml dry THF under a nitrogen atmosphere. After stirring for 20 minutes a solution of 2,4-dichlorobenzonitrile (3.65 g, 21.2 mmol) in 25 ml THF was added. The reaction mixture was stirred for 20 hours at room temperature. Water (50 ml) was carefully added. Extraction with EtOAc (2×100 ml), drying (Na₂SO₄), filtration and evaporation to dryness afforded 7.7 g (98%) of the title compound.

### Step 2

### 1-(4-Benzyloxyphenyl)-2-(2,4-dichlorophenyl)-5-methyl-1H-imidazole-4-carboxylic acid ethyl ester

To N-(4-benzyloxyphenyl)-2,4-dichlorobenzamidine (6.88 g, 18.5 mmol) dissolved in 50 ml THF was added potassium carbonate (2.56 g, 18.5 mmol) and the suspension was stirred for 10 minutes. Ethyl-3-bromo-2-oxobutanoate (4.65 g, 22.2 mmol) was added dropwise over 1 hour, and the mixture was stirred for 66 hours at room temperature. The solution was filtered and evaporated to dryness. The residue was dissolved in acetic acid and refluxed for 1 hour. The mixture was cooled to room temperature, 100 ml water added and the product extracted with EtOAc (2×200 ml). The combined organic phases were washed with saturated sodium hydrogen carbonate, dried (Na₂SO₄), filtered and concentrated. Flash chromatography (silica, hexane:EtOAc 70:30, 60:40) afforded 5.75 g (65%) of the title compound as a pale yellow solid.

¹H NMR (CDCl₃): δ 7.5-7.2 (8H, m), 7.1-6.9 (4H, m), 5.1 (2H, s), 4.5 (2H, q), 2.5 (3H, s), 1.5 (3H, t), MS m/z 504 (M+Na), 985 (2 M+Na)

### Step 3

1-(4-Benzyloxyphenyl)-2-(2,4-dichlorophenyl)-5-methyl-1H-imidazole-4-carboxylic acid To a suspension of 1-(4-benzyloxyphenyl)-2-(2,4-dichlorophenyl)-5-methyl-1H-imidazole-4-carboxylic acid ethyl ester (3.62 g, 7.5 mmol) in 60 ml methanol was added potassium hydroxide (4.05 g, 72 mmol) in water (20 ml), and the reaction mixture was boiled under reflux for 2 hours. The mixture was cooled to room temperature, acidified to pH˜2 with 1 M HCl and extracted with ethyl acetate (2×200 ml). The combined organic phases were dried (Na₂SO₄), filtered and concentrated to give 3.38 g (99%) of the title compound.

### Step 4

### 1-[4-(benzyloxy)phenyl]-2-(2,4-dichlorophenyl)-5-methyl-N-[6-(trifluoromethyl)pyridin-3-yl]-1H-imidazole-4-carboxamide

1-[4-(benzyloxy)phenyl]-2-(2,4-dichlorophenyl)-5-methyl-1H-imidazole-4-carboxylic acid (1000 mg, 2.21 mmol) was suspended in DCM (15 ml) and oxalyl chloride (1400 mg, 11.03 mmol) was added at rt followed by one drop of DMF. The mixture was stirred at rt for 5 min whereafter the solvents were evaporated under reduced pressure. A mixture of 6-trifluoromethyl-pyridin-3-ylamine (407 mg, 2.51 mmol), TEA (360 mg, 3.56 mmol) and DCM (7 ml) was added dropwise to the acid chloride suspended in DCM (8 ml). The reaction mixture was stirred at rt for 1.5 h. DCM was added and the resulting mixture was washed with HCl (10%, aq) and brine, dried (MgSO₄), filtered and evaporated to yield the product (crude 1317 mg).

¹H NMR (400 MHz, CDCl₃) δ 9.50 (1H, s), 8.82 (1H, s), 8.55 (1H, d), 7.65 (1H, d), 7.39-6.90 (12H, m), 5.03 (2H, s), 2.50 (3H, s). MS m/z 597 (M+H)⁺.

### Step 5

### 2-(2,4-dichlorophenyl)-1-(4-hydroxyphenyl)-5-methyl-N-[6-(trifluoromethyl)pyridin-3-yl]-1H-imidazole-4-carboxamide

1-[4-(benzyloxy)phenyl]-2-(2,4-dichlorophenyl)-5-methyl-N-[6-(trifluoromethyl)pyridin-3-yl]-1H-imidazole-4-carboxamide (1169 mg, 1.96 mmol) was suspended in DCM (6 ml) and dimethyl sulfide (1216 mg, 19.57 mmol) was added followed by boron trifluoride (2777 mg, 19.57 mmol). The reaction mixture was stirred at rt for 31 h (dark). Water and DCM were added and the phases separated. The organic phase was washed with water (×4) and evaporated. The product was redissolved in MeOH and stirred at rt for 20 h before water was added and the MeOH removed under reduced pressure. The resulting mixture was extracted with diethylether (×2) and the combined organic phase was washed with brine, dried (MgSO₄), filtered and evaporated to yield the product (crude 776 mg).

¹H NMR (400 MHz, CDCl₃) δ 9.29 (1H, s), 8.75 (1H, s), 8.54 (1H, d), 7.64 (1H, d), 7.33 (1H, s), 7.24-7.20 (2H, m), 6.97-6.77 (4H, m), 5.51 (1H, br), 2.48 (3H, s). MS m/z 507 (M+H)⁺.

### Step 6

### 4-[2-(2,4-dichlorophenyl)-5-methyl-4-({[6-(trifluoromethyl)pyridin-3-yl]amino}carbonyl)-1H-imidazol-1-yl]phenyl 3,3,3-trifluoropropane-1-sulfonate

2-(2,4-dichlorophenyl)-1-(4-hydroxyphenyl)-5-methyl-N-[6-(trifluoromethyl)pyridin-3-yl]-1H-imidazole-4-carboxamide (150 mg, 0.30 mmol) was suspended in dry DCM (2 ml) and TEA (39 mg, 0.38 mmol) was added at rt. The resulting mixture was cooled to −78° C. and 3,3,3-trifluoro-propane-1-sulfonyl chloride (91 mg, 0.46 mmol) in 0.5 ml dry DCM was added dropwise. Stirred at −78° C. for 70 min. The reaction mixture was washed with water and evaporated. The product was purified by HPLC (30=>100% ACN in 0.1 M ammonium acetate during 40 min) to yield the product as a white solid (131 mg, yield over 3 steps 52%). ¹H NMR (400 MHz, CDCl₃) δ 9.29 (1H, s), 8.77 (1H, s), 8.56 (1H, d), 7.66 (1H, d), 7.35-7.19 (7H, m), 3.51 (2H, m), 2.79 (2H, m), 2.53 (3H, s). HRMS Calcd for [C₂₆H₁₈Cl₂F₆N₄O₄S+H]⁺: 667.041. Found: 667.039.

### Example 2

### 4-(2-(2,4-dichlorophenyl)-5-methyl-4-{[(5-methylpyridin-2-yl)amino]carbonyl}-1H-imidazol-1-yl)phenyl 3,3,3-trifluoropropane-1-sulfonate

### Step 1

### 1-[4-(benzyloxy)phenyl]-2-(2,4-dichlorophenyl)-5-methyl-N-(5-methylpyridin-2-yl)-1H-imidazole-4-carboxamide

1-[4-(benzyloxy)phenyl]-2-(2,4-dichlorophenyl)-5-methyl-1H-imidazole-4-carboxylic acid, prepared as in Ex. 1, Step 3 (3000 mg, 6.62 mmol) was suspended in DCM (70 ml) and oxalyl chloride (4200 mg, 33.09 mmol) was added at rt followed by one drop of DMF. The mixture was stirred at rt for 5 min whereafter the solvents were evaporated under reduced pressure. A mixture of 5-methyl-pyridin-2-ylamine (816 mg, 7.54 mmol), TEA (890 mg, 8.80 mmol) and DCM (20 ml) was added dropwise to the acid chloride suspended in DCM (20 ml). The reaction mixture was stirred at rt for 30 min. DCM was added and the resulting mixture was washed with HCl (10%, aq) and brine, dried (MgSO₄), filtered and evaporated. The residue was purified by flash chromatography (20-30% EtOAc in heptane) to yield the product as a white solid (980 mg, 27%).

¹H NMR (400 MHz, Pyridine) δ 10.11 (1H, s), 8.52 (1H, s), 8.04 (1H, s), 7.40-6.88 (13H, m), 4.80 (2H, s), 2.39 (3H, s), 1.88 (3H, s). MS m/z 543 (M+H)⁺.

### Step 2

### 2-(2,4-dichlorophenyl)-1-(4-hydroxyphenyl)-5-methyl-N-(5-methylpyridin-2-yl)-1H-imidazole-4-carboxamide

1-[4-(benzyloxy)phenyl]-2-(2,4-dichlorophenyl)-5-methyl-N-(5-methylpyridin-2-yl)-1H-imidazole-4-carboxamide (958 mg, 1.76 mmol) was suspended in 33% HBr in acetic acid (25 ml). The reaction mixture was stirred at rt, in the dark, for 1 h. Ethanol was added and the solvents were evaporated at reduced pressure. The residue was redissolved in MeOH and neutralised with NaHCO₃ (1M, aq). The solvent was evaporated and the mixture redissolved in water/DCM. The phases were separated and the organic phase was washed with brine, dried (MgSO₄), filtered and evaporated to yield the product (772 mg, 97%).

¹H NMR (400 MHz, Pyridine) δ 10.12 (1H, s), 8.52 (1H, s), 8.03 (1H, s), 7.40-6.89 (8H, m), 2.42 (3H, s), 1.88 (3H, s). MS m/z 453 (M+H)⁺.

### Step 3

### 4-(2-(2,4-dichlorophenyl)-5-methyl-4-{[(5-methylpyridin-2-yl)amino]carbonyl}-1H-imidazol-1-yl)phenyl 3,3,3-trifluoropropane-1-sulfonate

2-(2,4-Dichlorophenyl)-1-(4-hydroxyphenyl)-5-methyl-N-(5-methylpyridin-2-yl)-1H-imidazole-4-carboxamide (150 mg, 0.33 mmol) was suspended in dry DCM (2 ml) and TEA (44 mg, 0.43 mmol) was added at rt. The resulting mixture was cooled to −78° C. and 3,3,3-trifluoro-propane-1-sulfonyl chloride (94 mg, 0.48 mmol) in 0.5 ml dry DCM was added dropwise. Stirred at −78° C. for 80 min. The reaction mixture was washed with water and evaporated. The product was purified by HPLC (30=>100% ACN in 0.1 M ammonium acetate during 40 min) to yield the product as a white solid (132 mg, 65%).

¹H NMR (400 MHz, CDCl₃) δ 9.63 (1H, s), 8.23 (1H, d), 8.11 (1H, s), 7.51 (1H, d), 7.32-7.17 (7H, m), 3.50 (2H, m), 2.79 (2H, m), 2.53 (3H, s), 2.28 (3H, s). HRMS Calcd for [C₂₆H₂₁Cl₂F₃N₄O₄S+H]⁺: 613.069. Found: 613.070.

### Example 3

### 4-(2-(2,4-dichlorophenyl)-4-{[(6-fluoropyridin-3-yl)amino]carbonyl}-5-methyl-1H-imidazol-1-yl)phenyl 3,3,3-trifluoropropane-1-sulfonate

### Step 1

### 1-[4-(benzyloxy)phenyl]-2-(2,4-dichlorophenyl)-N-(6-fluoropyridin-3-yl)-5-methyl-1H-imidazole-4-carboxamide

1-[4-(benzyloxy)phenyl]-2-(2,4-dichlorophenyl)-5-methyl-1H-imidazole-4-carboxylic acid, prepared as Ex. 1, Step 3 (1000 mg, 2.21 mmol) was suspended in DCM (15 ml) and oxalyl chloride (1400 mg, 11.03 mmol) was added at rt followed by one drop of DMF. The mixture was stirred at rt for 5 min whereafter the solvents were evaporated under reduced pressure. A mixture of 6-fluoro-pyridin-3-ylamine (297 mg, 2.65 mmol), TEA (313 mg, 3.09 mmol) and DCM (7 ml) was added dropwise to the acid chloride suspended in DCM (8 ml). The reaction mixture was stirred at rt for 75 min. DCM was added and the resulting mixture was washed with HCl (10%, aq) and brine, dried (MgSO₄), filtered and evaporated to yield the product (crude 1190 mg).

¹H NMR (400 MHz, CDCl₃) δ 9.24 (1H, s), 8.39-8.33 (2H, m), 7.39-6.89 (13H, m), 5.02 (2H, s), 2.49 (3H, s). MS m/z 547 (M+H)⁺.

### Step 2

### 2-(2,4-dichlorophenyl)-N-(6-fluoropyridin-3-yl)-1-(4-hydroxyphenyl)-5-methyl-1H-imidazole-4-carboxamide

1-[4-(benzyloxy)phenyl]-2-(2,4-dichlorophenyl)-N-(6-fluoropyridin-3-yl)-5-methyl-1H-imidazole-4-carboxamide (1151 mg, 2.10 mmol) was suspended in 33% HBr in acetic acid (25 ml). The reaction mixture was stirred at rt, in the dark, for 2 h 30 min. Ethanol was added and the solvents were evaporated at reduced pressure. The residue was redissolved in MeOH and neutralised with NaHCO₃ (1M, aq). The solvent was evaporated and the mixture redissolved in water/DCM. The phases were separated and the organic phase was washed with brine, dried (MgSO₄), filtered and evaporated. The product was purified by HPLC (30=>60% ACN in 0.1 M ammonium acetate during 40 min, then 100% ACN) to yield the product as a white solid (519 mg, yield over 2 steps 53%).

¹H NMR (400 MHz, CDCl₃) δ 9.14 (1H, s), 8.37-8.30 (2H, m), 7.34 (1H, s), 7.25-7.20 (2H, m), 6.96-6.90 (3H, m), 6.79-6.77 (2H, m), 2.48 (3H, s). MS m/z 457 (M+H)⁺.

### Step 3

### 4-(2-(2,4-dichlorophenyl)-4-{[(6-fluoropyridin-3-yl)amino]carbonyl}-5-methyl-1H-imidazol-1-yl)phenyl 3,3,3-trifluoropropane-1-sulfonate

2-(2,4-dichlorophenyl)-N-(6-fluoropyridin-3-yl)-1-(4-hydroxyphenyl)-5-methyl-1H-imidazole-4-carboxamide (150 mg, 0.33 mmol) was suspended in dry DCM (2 ml) and TEA (43 mg, 0.43 mmol) was added at rt. The resulting mixture was cooled to −78° C. and 3,3,3-trifluoro-propane-1-sulfonyl chloride (90 mg, 0.46 mmol) in 0.5 ml dry DCM was added dropwise. Stirred at −78° C. for 2 h 30 min, adding more of 3,3,3-trifluoro-propane-1-sulfonyl chloride (14 mg, 0.07 mmol) after 2 h. The reaction mixture was washed with water and evaporated. The product was purified by HPLC (30=>100% ACN in 0.1 M ammonium acetate during 35 min) to yield the product as a white solid (133 mg, 66%). ¹H NMR (400 MHz, CDCl₃) δ 9.10 (1H, s), 8.36-8.33 (2H, m), 7.35-7.18 (7H, m), 6.94-6.91 (1H, m), 3.53-3-49 (2H, m), 2.85-2.73 (2H, m), 2.53 (3H, s). HRMS Calcd for [C₂₅H₁₈Cl₂F₄N₄O₄S+H]⁺: 617.044. Found: 617.047.

### Example 4

### 4-[2-(2,4-dichlorophenyl)-4-({[(1S,2R)-2-hydroxycyclohexyl]amino}carbonyl)-5-methyl-1H-imidazol-1-yl]phenyl 3,3,3-trifluoropropane-1-sulfonate and 4-[2-(2,4-dichlorophenyl)-4-({[(1R,2S)-2-hydroxycyclohexyl]amino}carbonyl)-5-methyl-1H-imidazol-1-yl]phenyl 3,3,3-trifluoropropane-1-sulfonate

### Step 1

### 1-[4-(benzyloxy)phenyl]-2-(2,4-dichlorophenyl)-N-[(1S,2R)-2-hydroxycyclohexyl]-5-methyl-1H-imidazole-4-carboxamide and 1-[4-(benzyloxy)phenyl]-2-(2,4-dichlorophenyl)-N-[(1R,2S)-2-hydroxycyclohexyl]-5-methyl-1H-imidazole-4-carboxamide

1-[4-(benzyloxy)phenyl]-2-(2,4-dichlorophenyl)-5-methyl-1H-imidazole-4-carboxylic acid, prepared as Ex. 1, Step 3 (2000 mg, 4.41 mmol) was suspended in DCM (100 ml) and oxalyl chloride (2850 mg, 22.45 mmol) was added at rt followed by one drop of DMF. The mixture was stirred at rt for 20 min whereafter the solvents were evaporated under reduced pressure. The acid chloride suspended in DCM (10 ml) was added dropwise to a mixture of cis-2-aminocyclohexanol hydrochloride (816 mg, 5.38 mmol), 1M NaOH (aq) (30 ml) and DCM (30 ml). Stirred at rt for 2 h and water was added and the phases were separated. The organic phase was washed with HCl (0.1 M, aq) and brine, dried (MgSO₄), filtered and evaporated yield the racemic mixture (2398 mg, 99%).

¹H NMR (400 MHz, CDCl₃) δ 7.46-7.17 (8H, m), 6.99-6.89 (4H, m), 5.01 (2H, s), 4.12 (1H, m), 4.00 (1H, m), 2.43 (3H, s), 1.76-1.61 (6H, m), 1.39 (2H, m). MS m/z 550 (M+H)⁺.

### Step 2

### 2-(2,4-dichlorophenyl)-N-[(1S,2R)-2-hydroxycyclohexyl]-1-(4-hydroxyphenyl)-5-methyl-1H-imidazole-4-carboxamide and 2-(2,4-dichlorophenyl)-N-[(1R,2S)-2-hydroxycyclohexyl]-1-(4-hydroxyphenyl)-5-methyl-1H-imidazole-4-carboxamide

The racemic mixture of 1-[4-(benzyloxy)phenyl]-2-(2,4-dichlorophenyl)-N-[(1S,2R)-2-hydroxycyclohexyl]-5-methyl-1H-imidazole-4-carboxamide and 1-[4-(benzyloxy)phenyl]-2-(2,4-dichlorophenyl)-N-[(1R,2S)-2-hydroxycyclohexyl]-5-methyl-1H-imidazole-4-carboxamide (2384 mg, 4.33 mmol) was suspended in 33% HBr in acetic acid (50 ml). The reaction mixture was stirred at rt, in the dark, for 1 h. Ethanol was added and the solvents were evaporated at reduced pressure. The residue was redissolved in MeOH and neutralised with NaHCO₃ (1M, aq). The solvent was evaporated and the mixture redissolved in water/DCM. The phases were separated and the organic phase was washed with brine, dried (MgSO₄), filtered and evaporated. The residue was redissolved in MeOH and one spoon of K₂CO₃ was added, stirred at rt for 1 h before the solvent was evaporated. Resuspended in DCM and washed with HCl (10%, aq), evaporated the solvents. Redissolved in THF, dried (MgSO₄), filtered and evaporated to yield the racemic mixture (crude 2103 mg).

¹H NMR (400 MHz, THF) δ 8.67 (1H, d), 7.68 (1H, d), 7.57 (1H, s), 7.28-7.09 (3H, m), 6.80 (2H, d), 3.96-3.89 (2H, m), 2.49 (3H, s), 1.86-1.35 (8H, m). MS m/z 460 (M+H)⁺.

### Step 3

### 4-[2-(2,4-dichlorophenyl)-4-({[(1S,2R)-2-hydroxycyclohexyl]amino}carbonyl)-5-methyl-1H-imidazol-1-yl]phenyl 33,33-trifluoropropane-1-sulfonate and 4-[2-(2,4-dichlorophenyl)-4-({[(1R,2S)-2-hydroxycyclohexyl]amino}carbonyl)-5-methyl-1H-imidazol-1-yl]phenyl 3,3,3-trifluoropropane-1-sulfonate

The mixture of 2-(2,4-dichlorophenyl)-N-[(1S,2R)-2-hydroxycyclohexyl]-1-(4-hydroxyphenyl)-5-methyl-1H-imidazole-4-carboxamide and 2-(2,4-dichlorophenyl)-N-[(1R,2S)-2-hydroxycyclohexyl]-1-(4-hydroxyphenyl)-5-methyl-1H-imidazole-4-carboxamide (2000 mg, 4.34 mmol) was suspended in dry DCM (30 ml) and TEA (440 mg, 4.34 mmol) was added at rt. The resulting mixture was cooled to −78° C. and 3,3,3-trifluoro-propane-1-sulfonyl chloride (854 mg, 4.34 mmol) was added dropwise. Stirred at −78° C. for 2 h 20 min, adding more of the TEA (2×(73 mg, 0.72 mmol)) and 3,3,3-trifluoro-propane-1-sulfonyl chloride (2×(110 mg, 0.56 mmol)) after 1 h and 2 h. The reaction mixture was washed with water and evaporated. The product was purified by HPLC (30=>100% ACN in 0.1 M ammonium acetate during 40 min) to yield the product as a white solid (1311 mg, yield over 2 steps 51%).

¹H NMR (400 MHz, CDCl₃) δ 7.45 (1H, d), 7.31-7.13 (7H, m), 4.13 (1H, m), 4.00 (1H, m), 3.51-3.47 (2H, m), 2.81-2.74 (2H, m), 2.46 (3H, s), 1.78-1.40 (8H, m). HRMS Calcd for [C₂₆H₂₆Cl₂F₃N₃O₅S+H]⁺: 620.100. Found: 620.103.

### Example 5

### 4-[2-(2,4-dichlorophenyl)-4-({[(1 S,2S)-2-hydroxycyclohexyl]amino}carbonyl)-5-methyl-1H-imidazol-1-yl]phenyl 3,3,3-trifluoropropane-1-sulfonate and 4-[2-(2,4-dichlorophenyl)-4-({[(1R,2R)-2-hydroxycyclohexyl]amino}carbonyl)-5-methyl-1H-imidazol-1-yl]phenyl 3,3,3-trifluoropropane-1-sulfonate

### Step 1

### 1-[4-(benzyloxy)phenyl]-2-(2,4-dichlorophenyl)-N-[(1S,2S)-2-hydroxycyclohexyl]-5-methyl-1H-imidazole-4-carboxamide and 1-[4-(benzyloxy)phenyl]-2-(2,4-dichlorophenyl)-N-[(1R,2R)-2-hydroxycyclohexyl]-5-methyl-1H-imidazole-4-carboxamide

1-[4-(benzyloxy)phenyl]-2-(2,4-dichlorophenyl)-5-methyl-1H-imidazole-4-carboxylic acid, prepared as in Ex. 1, Step 3 (2000 mg, 4.41 mmol) was suspended in DCM (100 ml) and oxalyl chloride (2800 mg, 22.06 mmol) was added at rt followed by one drop of DMF. The mixture was stirred at rt for 35 min whereafter the solvents were evaporated under reduced pressure. The acid chloride suspended in DCM (10 ml) was added dropwise to a mixture of trans-2-aminocyclohexanol hydrochloride (802 mg, 5.29 mmol), 1M NaOH (aq) (30 ml) and DCM (30 ml). Stirred at rt for 2 h whereafter water/DCM were added and the phases were separated. The organic phase was washed with HCl (10%, aq) and brine, dried (MgSO₄), filtered and evaporated yield a recemic mixture (crude 2686 mg).

¹H NMR (400 MHz, CDCl₃) δ 7.38-7.24 (8H, m), 7.02-6.93 (4H, m), 5.02 (2H, s), 3.76 (1H, m), 3.55 (1H, m), 2.47 (3H, s), 2.08 (2H, m), 1.71 (2H, br), 1.39-1.23 (4H, m). MS m/z 550 (M+H)⁺.

### Step 2

### 2-(2,4-dichlorophenyl)-N-[(1S,2S)-2-hydroxycyclohexyl]-1-(4-hydroxyphenyl)-5-methyl-1H-imidazole-4-carboxamide and 2-(2,4-dichlorophenyl)-N-[(1R,2R)-2-hydroxycyclohexyl]-1-(4-hydroxyphenyl)-5-methyl-1H-imidazole-4-carboxamide

The mixture of 1-[4-(benzyloxy)phenyl]-2-(2,4-dichlorophenyl)-N-[(1S,2S)-2-hydroxycyclohexyl]-5-methyl-1H-imidazole-4-carboxamide and 1-[4-(benzyloxy)phenyl]-2-(2,4-dichlorophenyl)-N-[(1R,2R)-2-hydroxycyclohexyl]-5-methyl-1H-imidazole-4-carboxamide (2680 mg, 4.87 mmol) was suspended in 33% HBr in acetic acid (60 ml). The reaction mixture was stirred at rt, in the dark, for 1 h 20 min. Ethanol was added and the solvents were evaporated at reduced pressure. The residue was redissolved in MeOH and neutralised with NaHCO₃ (1M, aq). One spoon of K₂CO₃ was added and the mixture was stirred at rt for 1 h. The solvent was evaporated and the resulting mixture extracted with toluene followed by THF. The combined organic phase was washed with HCl (10%, aq) and brine, dried (MgSO₄), filtered and evaporated. The product was purified by HPLC (30=>100% ACN in 0.1 M ammonium acetate during 40 min) to yield the product as a white solid (829 mg, yield over 2 steps 41%). ¹H NMR (400 MHz, CDCl₃) δ 7.31-7.18 (3H, m), 6.82-6.73 (4H, m), 4.60 (1H, br), 3.79 (1H, m), 3.46 (1H, m), 2.37 (3H, s), 2.09-1.98 (2H, m), 1.74-1.71 (2H, m), 1.43-1.20 (4H, m). MS m/z 460 (M+H)⁺.

### Step 3

### 4-[2-(2,4-dichlorophenyl)-4-({[(1S,2S)-2-hydroxycyclohexyl]amino}carbonyl)-5-methyl-1H-imidazol-1-yl]phenyl 3,3,3-trifluoropropane-1-sulfonate and 4-[2-(2,4-dichlorophenyl)-4-({[(1R,2R)-2-hydroxycyclohexyl]amino}carbonyl)-5-methyl-1H-imidazol-1-yl]phenyl 3,3,3-trifluoropropane-1-sulfonate

2-(2,4-dichlorophenyl)-N-[(1S,2S)-2-hydroxycyclohexyl]-1-(4-hydroxyphenyl)-5-methyl-1H-imidazole-4-carboxamide and 2-(2,4-dichlorophenyl)-N-[(1R,2R)-2-hydroxycyclohexyl]-1-(4-hydroxyphenyl)-5-methyl-1H-imidazole-4-carboxamide (829 mg, 1.80 mmol) was suspended in dry DCM (10 ml) and TEA (182 mg, 1.80 mmol) was added at rt. The resulting mixture was cooled to −78° C. and 3,3,3-trifluoropropane-1-sulfonyl chloride (354 mg, 1.80 mmol) in 1 ml dry DCM was added dropwise. Stirred at −78° C. for 1 h. The reaction mixture was washed with water and evaporated. The product was purified by HPLC (30=>100% ACN in 0.1 M ammonium acetate during 40 min) to yield the product as a white solid (710 mg, 64%). ¹H NMR (400 MHz, CDCl₃) δ 7.31-7.14 (7H, m), 3.79 (1H, m), 3.51-3.41 (3H, m), 2.81-2.75 (2H, m), 2.47 (3H, s), 2.07-1.99 (2H, m), 1.73-1.71 (2H, m), 1.38-1.30 (4H, m). HRMS Calcd for [C₂₆H₂₆Cl₂F₃N₃O₅S+H]⁺: 620.100. Found: 620.101.

### Example 6

### 4-{2-(2,4-dichlorophenyl)-5-methyl-4-[(piperidin-1-ylamino)carbonyl]-1H-imidazol-1-yl}phenyl 3-fluoropropane-1-sulfonate

### Step 1

### 1-(4-Benzyloxyphenyl)-2-(2,4-dichlorophenyl)-5-methyl-1H-imidazole-4-carboxylic acid piperidin-1-ylamide

A solution of 1-(4-benzyloxyphenyl)-2-(2,4-dichlorophenyl)-5-methyl-1H-imidazole-4-carboxylic acid, from Ex 1, Step 3 (3.38 g, 7.5 mmol) in 60 ml CH₂Cl₂ was added 3 drops of DMF followed by oxalyl chloride (1.3 ml, 14.9 mmol). The mixture was refluxed for 2 hours, cooled to room temperature and evaporated to dryness. The residue was dissolved in 50 ml CH₂Cl₂ and cooled to 0° C. Triethylamine (2.1 ml, 14.9 mmol) was added followed by 1-aminopiperidine (0.9 ml, 8.2 mmol) and the mixture was stirred at room temperature for 2 hours. Water (300 ml) was added, the mixture extracted with CH₂Cl₂ (3×100 ml), dried (Na₂SO₄), filtered and concentrated. Flash chromatography (silica, hexane:EtOAc 1:2, EtOAc) afforded 2.94 g (74%) of the title compound as a white solid.

¹H NMR (CDCl₃): δ 7.5-7.2 (8H, m), 7.1-6.9 (4H, m), 5.1 (2H, s), 3.0-2.7 (4H, m), 2.5 (3H, s), 1.9-1.7 (4H, m), 1.6-1.4 (2H, m). MS m/z 558 (M+Na). HPLC: 96.5%.

### Step 2

### 2-(2,4-Dichlorophenyl)-1-(4-hydroxyphenyl)-5-methyl-1H-imidazole-4-carboxylic acid piperidin-1-ylamide

1-(4-Benzyloxyphenyl)-2-(2,4-dichlorophenyl)-5-methyl-1H-imidazole-4-carboxylic acid piperidin-1-ylamide (2.78 g, 5.2 mmol) was dissolved in 80 ml CH₂Cl₂ and cooled to 0° C. Boron tribromide solution (1 M in CH₂Cl₂, 10.4 ml, 10.4 mmol) was added dropwise and the reaction mixture was stirred at room temperature for 1 hour. Water (200 ml) was added and the solution extracted with EtOAc (3×200 ml). The combined organic phases were dried (Na₂SO₄), filtered and concentrated. Flash chromatography (silica, hexane:EtOAc 1:3, EtOAc) afforded 1.34 g (58%) of the title compound as a white solid.

¹H NMR (CDCl₃): δ 8.6 (1H, bs), 7.4-7.1 (3H, m), 7.0-6.9 (4H, m), 3.0-2.8 (4H, m), 2.5 (3H, s), 1.8-1.6 (4H, m), 1.5-1.3 (2H, m).

### Step 3

### 4-{2-(2,4-dichlorophenyl)-5-methyl-4-[(piperidin-1-ylamino)carbonyl]-1H-imidazol-1-yl}phenyl 3-fluoropropane-1-sulfonate

2-(2,4-dichlorophenyl)-1-(4-hydroxyphenyl)-5-methyl-N-piperidin-1-yl-1H-imidazole-4-carboxamide (200 mg, 0.45 mmol) was suspended in dry DCM (3 ml) and TEA (45 mg, 0.45 mmol) was added at rt. The resulting mixture was cooled to −78° C. and 3-fluoro-propane-1-sulfonyl chloride (72 mg, 0.45 mmol) in 0.5 ml dry DCM was added dropwise. After 1 h 40 min at −78° C. was added 3-fluoro-propane-1-sulfonyl chloride (72 mg, 0.45 mmol) and after totally 4 h 40 min was added TEA (55 mg, 0.54 mmol). The reaction was continued over night from −78° C.=>rt. Cooled to 0° C. and added TEA (55 mg, 0.54 mmol) followed by 3-fluoro-propane-1-sulfonyl chloride (72 mg, 0.45 mmol) after totally 19 h. After totally 20 h the reaction mixture was washed with water and evaporated. The product was purified by HPLC (30=>100% ACN in 0.1 M ammonium acetate during 40 min) to yield the product as a white solid (160 mg, 63%).

¹H NMR (400 MHz, CDCl₃) δ 7.88 (1H, br), 7.29-7.10 (7H, m), 4.58 (2H, dt), 3.41 (2H, t), 2.82 (4H, br), 2.45 (3H, s), 2.39-2.25 (2H, m), 1.72 (4H, br), 1.40 (2H, br). HRMS Calcd for [C₂₅H₂₇Cl₂FN₄O₄S+H]⁺: 569.119. Found: 569.123.

### Example 7

### 4-[2-(2,4-dichlorophenyl)-5-methyl-4-({[4-(trifluoromethoxy)phenyl]amino}carbonyl)-1H-imidazol-1-yl]phenyl 3,3,3-trifluoropropane-1-sulfonate

### Step 1

### 1-[4-(benzyloxy)phenyl]-2-(2,4-dichlorophenyl)-5-methyl-N-[4-(trifluoromethoxy)phenyl]-1H-imidazole-4-carboxamide

1-[4-(benzyloxy)phenyl]-2-(2,4-dichlorophenyl)-5-methyl-1H-imidazole-4-carboxylic acid, prepared as in Ex. 1, Step 3 (1000 mg, 2.21 mmol) was suspended in DCM (15 ml) and oxalyl chloride (1400 mg, 11.03 mmol) was added at rt followed by one drop of DMF. The mixture was stirred at rt for 15 min whereafter the solvents were evaporated under reduced pressure. A mixture of 4-trifluoromethoxy-phenylamine (469 mg, 2.65 mmol), TEA (313 mg, 3.09 mmol) and DCM (5 ml) was added dropwise to the acid chloride suspended in DCM (15 ml). The reaction mixture was stirred at rt for 2 h 10 min. DCM was added and the resulting mixture was washed with HCl (10%, aq) and brine, dried (MgSO₄), filtered and evaporated to yield the product (crude 1415 mg).

¹H NMR (400 MHz, CDCl₃) δ 9.37 (1H, br), 7.76-7.74 (2H, m), 7.39-7.16 (10H, m), 7.05-6.93 (4H, m), 5.03 (2H, s), 2.50 (3H, s). MS m/z 612 (M+H)⁺.

### Step 2

### 2-(2,4-dichlorophenyl)-1-(4-hydroxyphenyl)-5-methyl-N-[4-(trifluoromethoxy)phenyl]-1H-imidazole-4-carboxamide

1-[4-(benzyloxy)phenyl]-2-(2,4-dichlorophenyl)-5-methyl-N-[4-(trifluoromethoxy)-phenyl]-1H-imidazole-4-carboxamide (1346 mg, 2.20 mmol) was suspended in 33% HBr in acetic acid (25 ml). The reaction mixture was stirred at rt, in the dark, for 1 h. Ethanol was added and the solvents were evaporated at reduced pressure. The residue was redissolved in MeOH and neutralised with NaHCO₃ (1M, aq). The solvent was evaporated and the mixture redissolved in water/DCM. The phases were separated and the organic phase was washed with brine, dried (MgSO₄), filtered and evaporated to yield the product (crude 1102 mg).

¹H NMR (400 MHz, CDCl₃) δ 7.73-7.71 (2H, m), 7.39-7.16 (5H, m), 6.94-6.76 (4H, m), 2.45 (3H, s). MS m/z 522 (M+H)⁺.

### Step 3

### 4-[2-(2,4-dichlorophenyl)-5-methyl-4-({[4-(trifluoromethoxy)phenyl]amino}carbonyl)-1H-imidazol-1-yl]phenyl 3,3,3-trifluoropropane-1-sulfonate

2-(2,4-dichlorophenyl)-1-(4-hydroxyphenyl)-5-methyl-N-[4-(trifluoromethoxy)phenyl]-1H-imidazole-4-carboxamide (150 mg, 0.29 mmol) was suspended in dry DCM (2 ml) and TEA (38 mg, 0.37 mmol) was added at rt. The resulting mixture was cooled to −78° C. and 3,3,3-trifluoro-propane-1-sulfonyl chloride (79 mg, 0.40 mmol) in 0.5 ml dry DCM was added dropwise. Stirred at −78° C. for 70 min. The reaction mixture was washed with water and evaporated. The product was purified by HPLC (30=>100% ACN in 0.1 M ammonium acetate during 35 min) to yield the product as a white solid (84 mg, yield over 3 steps 43%). ¹H NMR (400 MHz, CDCl₃) δ 9.10 (1H, s), 7.72-7.70 (2H, m), 7.35-7.17 (9H, m), 3.52-3.48 (2H, m), 2.85-2.73 (2H, m), 2.53 (3H, s). HRMS Calcd for [C₂₇H₁₉Cl₂F₆N₃O₅S+H]⁺: 682.041. Found: 682.040.

### Example 8

### 3,3,3-Trifluoropropane-1-sulfonic acid 4-[2-(2,4-dichlorophenyl)-4-(3-hydroxypiperidin-1-ylcarbamoyl)-5-methylimidazol-1-yl]phenyl ester

### Step 1

### 1-(4-Benzyloxy-phenyl)-2-(2,4-dichloro-phenyl)-5-methyl-1H-imidazole-4-carboxylic acid (3-hydroxy-piperidin-1-yl)-amide

1-(4-Benzyloxy-phenyl)-2-(2,4-dichloro-phenyl)-5-methyl-1H-imidazole-4-carboxylic acid, prepared as in Ex. 1, Step 3 (752 mg, 1.66 mmol, 1 equiv.) and thionyl chloride (20 equiv) were mixed and the resulting mixture was refluxed for 1.5 h. Excess SOCl₂ was removed under reduced pressure and the residue was azeotroped with toluene. 3-Hydroxy-1-aminopiperidine (4 equiv.) was mixed with dichloromethane (15 ml) and THF (2 ml) and triethyl amine (8 equiv). The mixture was cooled to −20 degrees under a nitrogen atmosphere. A THF (5 ml) mixture of the acid chloride from above was added dropwise during 20 minutes. The resulting mixture was allowed to slowly warm to room temperature and stirred overnight. Aqueous NaOH (1M, 5 ml) and methanol (15 ml) were added and the mixture was heated to 40 degrees for 15 minutes. The reaction mixture was then diluted to 50 ml with dichloromethane and washed with water (2×20 ml) and brine (20 ml). The organic layer was dried (MgSO₄), filtered and concentrated under reduced pressure. The residue was purified by Horizon flash chromatography, 8% methanol in dichloromethane and then by reverse phase HPLC (Kromasil C8, 60% acetonitrile in water with 0.1M ammonium acetate). The product fraction was concentrated under reduced pressure and then dissolved in dichloromethane and washed with water several times and then brine. The organic layer was dried (MgSO₄), filtered and concentrated under reduced pressure to give the title compound (160 mg, 17% yield).

¹H-NMR (CDCl₃): 1.41-1.53 (m, 1H), 1.56-1.71 (m, 2H), 1.84-1.94 (m, 1H), 2.41 (s, 3H), 2.72-2.91 (m, 3H), 3.03-3.13 (m, 1H), 3.91-4.01 (m, 1H), 4.99 (s, 2H), 6.88 (d, 2H), 6.97 (d, 2H), 7.12-7.40 (m, 8H), 8.06 (s, 1H). MS: 551 (M+1).

### Step 2

### 2-(2,4-Dichloro-phenyl)-1-(4-hydroxy-phenyl)-5-methyl-1H-imidazole-4-carboxylic acid (3-hydroxy-piperidin-1-yl)-amide

1-(4-Benzyloxy-phenyl)-2-(2,4-dichloro-phenyl)-5-methyl-1H-imidazole-4-carboxylic acid (3-hydroxy-piperidin-1-yl)-amide (160 mg, 0.29 mmol, 1 equiv.) and dimethyl sulfide (5 equiv) were mixed in dichloromethane under nitrogen atmosphere. BF₃xOEt₂ (boron trifluoride etherate) (5 equiv.) was added dropwise and the resulting mixture was stirred for 4 days at ambient temperature while continuously adding small volumes of dichloromethane and 1,4-dioxane. Methanol was added and the mixture was stirred for 30 mins and the mixture was then concentrated under reduced pressure. The residue was dissolved in ethyl acetate (50 ml) and washed with water (2×20 ml) and brine (20 ml). The organic layer was dried (Na₂SO₄), filtered and concentrated under reduced pressure to give the title compound (127 mg, 95% yield) as a white solid.

MS: 461 (M+1).

### Step 3

### 3,3,3-Trifluoropropane-1-sulfonic acid 4-[2-(2,4-dichloro-phenyl)-4-(3-hydroxy-piperidin-1-ylcarbamoyl)-5-methyl-imidazol-1-yl]-phenyl ester

2-(2,4-Dichloro-phenyl)-1-(4-hydroxy-phenyl)-5-methyl-1H-imidazole-4-carboxylic acid (3-hydroxy-piperidin-1-yl)-amide (118 mg, 0.25 mmol, 1 equiv.) was dissolved in dichloromethane (1 ml), THF (1 ml) and triethylamine (1 equiv.) under a nitrogen atmosphere. The solution was cooled to −78 and a solution of 3,3,3-trifluoro-propane-1-sulfonyl chloride in dichloromethane (1 ml) was added slowly while monitoring the progress with LC-MS. The reaction mixture was quenched by addition of methanol. The reaction mixture was concentrated under reduced pressure. The residue was purified by reverse phase HPLC (Kromasil C8, 5-100% acetonitrile in water with 0.1M ammonium acetate) and by Horizon flash chromatography (8% methanol in dichloromethane). The product was freeze-dried to give the title compound (40 mg, 25% yield) as a white powder.

¹H-NMR (MeOH-d₄): 1.23-1.43 (m, 1H), 1.54-1.95 (m, 3H), 2.43 (s, 3H), 2.51-2.74 (m, 2H), 2.75-2.94 (m, 3H), 3.01-3.16 (m, 1H), 3.64-3.78 (m, 2H), 3.79-3.92 (m, 1H), 7.28-7.52 (m, 7H). MS: 621 (M+1). HPLC-UV: 99%.

### Example 9

### 3,3,3-Trifluoropropane-1-sulfonic acid 4-[2-(2,4-dichlorophenyl)-4-(4-hydroxypiperidin-1-ylcarbamoyl)-5-methyl-imidazol-1-yl]phenyl ester

### Step 1

### 1-(4-Benzyloxy-phenyl)-2-(2,4-dichloro-phenyl)-5-methyl-1H-imidazole-4-carboxylic acid (4-hydroxy-piperidin-1-yl)-amide

This compound was prepared as described for Ex 8, Step 1 using 4-hydroxy-1-aminopiperidine in stead of 3-hydroxy-1-aminopiperidine. The product was obtained as a semisolid (766 mg, 79% yield).

¹H-NMR (CDCl₃): 1.81 (m, 2H), 1.98 (m, 2H), 2.44 (s, 3H), 2.77 (m, 2H), 3.11 (m, 2H), 3.77 (m, 1H), 5.01 (s, 2H), 6.89 (d, 2H), 6.97 (d, 2H), 7.16-7.22 (m, 2H), 7.30-7.40 (m, 6H), 7.95 (s, 1H). MS: 551 (M+1).

### Step 2

### 2-(2,4-Dichloro-phenyl)-1-(4-hydroxy-phenyl)-5-methyl-1H-imidazole-4-carboxylic acid (4-hydroxy-piperidin-1-yl)-amide

This compound was prepared as described for Ex. 8 Step 2 and was obtained as a white solid (182 mg, 28% yield). MS: 461 (M+1).

### Step 3

### 3,3,3-Trifluoropropane-1-sulfonic acid 4-[2-(2,4-dichlorophenyl)-4-(4-hydroxypiperidin-1-ylcarbamoyl)-5-methyl-imidazol-1-yl]phenyl ester

This compound was prepared as described for Ex. 8, Step 3. The product was obtained as a white solid (38 mg, 18% yield) after freeze-drying.

¹H-NMR (MeOH-d₄): 1.46-1.65 (m, 2H), 1.66-1.82 (m, 2H), 2.25 (s, 3H), 2.46-2.77 (m, 4H), 2.78-2.93 (m, 2H), 3.42-3.60 (m, 3H), 7.09-7.35 (m, 7H). MS: 621 (M+1).

HPLC-UV: 99%

### Example 10

### (−)4-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate

The enantiomer was separated from Ex. 4 (1000 mg, 1.61 mmol) by Chiral chromatography (Chiralpak AD, heptane/IPA 80/20) to afford 484 mg (>99.9% ee) as white powder after freeze drying.

[α]_{D}²⁰=−10.0 (c 1.03, acetonitrile).

¹H NMR (400 MHz, CDCl₃) δ 7.45 (1H, d), 7.31-7.13 (7H, m), 4.13 (1H, m), 4.00 (1H, m), 3.51-3.47 (2H, m), 2.81-2.74 (2H, m), 2.46 (3H, s), 1.78-1.40 (8H, m).

HRMS Calcd for [C₂₆H₂₆Cl₂F₃N₃O₅S+H]⁺: 620.100. Found: 620.095.

### Example 11

### 2-(2,4-Dichlorophenyl)-5-methyl-1-[4-(3,3,3-trifluoropropoxy)phenyl]-1H-imidazole-4-carboxylic acid (2-hydroxycyclohexyl)amide

### Step 1 Ethyl 2-(2,4-dichlorophenyl-1-(4-hydroxyphenyl)-5-methyl-1H-imidazole-4-carboxylate

Ethyl 1-[4-(benzyloxy)phenyl]-2-(2,4-dichlorophenyl)-5-methyl-1H-imidazole-4-carboxylate, Ex 1, Step 2 (4.82 g, 10 mmol) was dissolved in 80 ml HBr (33% in acetic acid) and stirred overnight at room temperature with exclusion of light. The solvents were evaporated and the residue co-evaporated with ethanol. The residue was dissolved in ethanol, 5 ml of HCl in dioxane (4M) and MgSO4 was added and the resulting mixture heated under reflux for 2.5 h. The reaction mixture was cooled to room temperature, filtered and evaporated. The residue was dissolved in EtOAc and washed with water basified with triethylamine and then brine. The organic layer was dried over Na₂SO₄ and evaporated to give ethyl 2-(2,4-dichlorophenyl)-1-(4-hydroxyphenyl)-5-methyl-1H-imidazole-4-carboxylate (4.74 g) as a brown, viscous oil of sufficient purity for the next step.

¹H NMR (500 MHz, CDCl₃) δ 8.65 (br. s, 1H), 7.28-7.14 (m, 3H), 6.88 (d, J=8.6 Hz, 2H), 6.79 (d, J=8.6 Hz, 2H), 4.32 (q, J=7.1 Hz, 2H), 2.43 (s, 3H), 1.27 (t, J=7.1 Hz, 3H)

### Step 2 Ethyl 2-(2,4-dichlorophenyl)-5-methyl-1-[4-(3,33-trifluoropropoxy)phenyl]-1H-imidazole-4-carboxylate

Ethyl 2-(2,4-dichlorophenyl)-1-(4-hydroxyphenyl)-5-methyl-1H-imidazole-4-carboxylate (978 mg, 2.5 mmol), 3,3,3-trifluoro-1-propanol (428 mg, 3.75 mmol) and triphenylphosphine (984 mg, 3.75 mmol) were dissolved in anhydrous THF (12 ml). Then DEAD (1.72 ml of a ca. 40% solution in toluene, d=0.95, 3.75 mmol) was added. The resulting mixture warms and was stirred at room temperature for 30 h, then heated to 50 C overnight. After cooling to room temperature, additional 3,3,3-trifluoro-1-propanol (428 mg, 3.75 mmol) and triphenylphosphine (984 mg, 3.75 mmol) followed by di-tert-butyl azodicarboxylate (863 mg, 3.75 mmol) was added and the resulting mixture stirred at room temperature overnight. Again, additional 3,3,3-trifluoro-1-propanol (428 mg, 3.75 mmol) and triphenylphosphine (984 mg, 3.75 mmol) followed by di-tert-butyl azodicarboxylate (863 mg, 3.75 mmol) was added and the resulting mixture stirred at room temperature overnight. The solvents were evaporated, the residue purified by column chromatography (silica gel, hexanes/EtOAc, 10-50%) to yield ethyl 2-(2,4-dichlorophenyl)-5-methyl-1-[4-(3,3,3-trifluoropropoxy)phenyl]-1H-imidazole-4-carboxylate (880 mg, 1.72 mmol, 68%) as a yellowish foam of sufficient purity for the next transformation.

¹H NMR (500 MHz, CDCl₃) δ 7.22-7.16 (m, 3H), 7.01 (d, J=8.7 Hz, 2H), 6.83 (d, J=8.7 Hz, 2H), 4.40 (q, J=7.1 Hz, 2H), 4.22-4.10 (m, 2H), 2.66-2.54 (m, 2H), 2.40 (s, 3H), 1.40 (t, J=7.1 Hz, 3H)

### Step 3 2-(2,4-dichlorophenyl)-5-methyl-1-[4-(3,3,3-trifluoropropoxy)phenyl]-1H-imidazole-4-carboxylic acid

Ethyl 2-(2,4-dichlorophenyl)-5-methyl-1-[4-(3,3,3-trifluoropropoxy)phenyl]-1H-imidazole-4-carboxylate (880 mg, 1.72 mmol) was dissolved in a mixture of 15 ml THF and 15 ml EtOH, then KOH (1.07 g, 19 mmol) dissolved in 10 ml water was added and the resulting mixture stirred at 50 C. After 3 h 30 min the reaction mixture was cooled to room temperature and the solvents were evaporated. The residue was partitioned between DCM and 1N HCl and after phase separation the aqueous layer was extracted two more times with DCM. The combined organic layers were dried over MgSO₄ and evaporated to give 2-(2,4-dichlorophenyl)-5-methyl-1-[4-(3,3,3-trifluoropropoxy)phenyl]-1H-imidazole-4-carboxylic acid (714 mg, 1.55 mmol, 90%) as a yellowish foam.

¹H NMR (500 MHz, CDCl₃) δ 7.32-7.18 (m, 3H), 7.00 (d, J=8.7 Hz, 2H), 6.85 (d, J=8.7 Hz, 2H), 4.18-4.14 (m, 2H), 2.66-2.55 (m, 2H), 2.42 (s, 3H)

### Step 4 2-(2,4-dichlorophenyl)-5-methyl-1-[4-(3,3,3-trifluoropropoxy)phenyl]-1H-imidazole-4-carbonyl chloride

2-(2,4-dichlorophenyl)-5-methyl-1-[4-(3,3,3-trifluoropropoxy)phenyl]-1H-imidazole-4-carboxylic acid (643 mg, 1.4 mmol) was dissolved in 10 ml DCM, then oxalyl chloride (200 μl, 2.36 mmol) was added, followed by 10 μl DMF. The resulting mixture was stirred for 90 min at room temperature, then the solvents were evaporated and the residue dried in oil pump vacuum to give 2-(2,4-dichlorophenyl)-5-methyl-1-[4-(3,3,3-trifluoropropoxy)phenyl]-1H-imidazole-4-carbonyl chloride (690 mg, 1.37 mmol, 98%) as a yellowish foam which was used without further purification in the next step.

### Step 5 2-(2,4-dichlorophenyl)-N-[(cis)-2-hydroxycyclohexyl]-5-methyl-1-[4-(3,3,3-trifluoropropoxy)phenyl]-1H-imidazole-4-carboxamide

To a mixture of cis-2-aminocyclohexanol hydrochloride (0.3 mmol) and 100 μl pyridine in 1 ml DCM was added crude 2-(2,4-dichlorophenyl)-5-methyl-1-[4-(3,3,3-trifluoropropoxy)phenyl]-1H-imidazole-4-carbonyl chloride (96 mg, 0.2 mmol) in 1 ml DCM and the resulting mixture stirred at room temperature for 2 h 30 min. The reaction mixture was washed with 2 ml of sat. NaHCO₃ and after phase separation filtered through a phase separator. The solvents were evaporated and the residue purified by preparative HPLC eluting on a reverse-phase column with 5 to 100% acetonitrile in 0.1 M NH₄Ac to give 2-(2,4-dichlorophenyl)-N-[(cis)-2-hydroxycyclohexyl]-5-methyl-1-[4-(3,3,3-trifluoropropoxy)phenyl]-1H-imidazole-4-carboxamide (46 mg, 82 μmol, 41%) as a colorless solid.

¹H NMR (500 MHz, CDCl₃) δ 7.46 (d, J=8.2 Hz, 1H), 7.35-7.22 (m, 3H), 7.04 (d, J=8.7 Hz, 2H), 6.87 (d, J=8.7 Hz, 2H), 4.21-4.16 (m, 3H), 4.06-4.04 (m, 1H), 2.77 (s, 1H), 2.68-2.60 (m, 2H), 2.47 (s, 3H), 1.83-1.43 (m, 8H). HRMS Calcd for [C₂₆H₂₆Cl₂F₃N₃O₃+H]⁺: 556.1382. Found: 556.1413.

### Example 12

### (+)₄-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate

The enantiomer was separated from Ex. 4 (1000 mg, 1.61 mmol) by Chiral chromatography (Chiralpak AD, heptane/IPA 80/20) ti yield 444 mg (>99.9% ee) as white powder after freeze drying.

[α]_{D}²⁰+9.9 (c 1.02, acetonitrile)

¹H NMR (400 MHz, CDCl₃) δ 7.45 (1H, d), 7.31-7.13 (7H, m), 4.13 (1H, m), 4.00 (1H, m), 3.51-3.47 (2H, m), 2.81-2.74 (2H, m), 2.46 (3H, s), 1.78-1.40 (8H, m).

HRMS Calcd for [C₂₆H₂₆Cl₂F₃N₃O₅S+H]⁺: 620.100. Found: 620.095.

### Example 13

### (+)₄-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate

The enantiomer was separated from Ex. 5 (535 mg, 0.86 mmol) through Chiral chromatography (Chiralpak AD, heptane/IPA 85/15) to yield 207 mg (95.5% ee) as white powder after freeze drying.

[α]_{D}²⁰=+2.8 (c 1.05, acetonitrile)

¹H NMR (400 MHz, CDCl₃) δ 7.31-7.14 (7H, m), 3.79 (1H, m), 3.51-3.41 (3H, m), 2.81-2.75 (2H, m), 2.47 (3H, s), 2.07-1.99 (2H, m), 1.73-1.71 (2H, m), 1.38-1.30 (4H, m).

HRMS Calcd for [C₂₆H₂₆Cl₂F₃N₃O₅S+H]⁺: 620.100. Found: 620.103.

### Example 14

### (−)4-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate

The enantiomer was separated from Ex. 5 (535 mg, 0.86 mmol) by Chiral chromatography (Chiralpak AD, heptane/IPA 85/15) to afford 220 mg (95.6% ee) as white solid after freeze drying.

[α]_{D}²⁰=−2.9 (c 1.04, acetonitrile)

¹H NMR (400 MHz, CDCl₃) δ 7.31-7.14 (7H, m), 3.79 (1H, m), 3.51-3.41 (3H, m), 2.81-2.75 (2H, m), 2.47 (3H, s), 2.07-1.99 (2H, m), 1.73-1.71 (2H, m), 1.38-1.30 (4H, m).

HRMS Calcd for [C₂₆H₂₆Cl₂F₃N₃O₅S+H]⁺: 620.100. Found: 620.096.

### Example 15

### 4-[4-({[(1S,2R)-2-aminocyclohexyl]amino}carbonyl)-2-(2,4-dichlorophenyl)-5-methyl-1H-imidazol-1-yl]phenyl 3,3,3-trifluoropropane-1-sulfonate

### Step 1 N-[(1S,2R)-2-aminocyclohexyl]-1-[4-(benzyloxy)phenyl]-2-(2,4-dichlorophenyl)-5-methyl-1H-imidazole-4-carboxamide

1-[4-(benzyloxy)phenyl]-2-(2,4-dichlorophenyl)-5-methyl-1H-imidazole-4-carboxylic acid, prepared as in Ex. 1, Step 3 (2000 mg, 4.41 mmol) was suspended in DCM (50 ml) and oxalyl chloride (2800 mg, 22.06 mmol) was added at rt followed by one drop of DMF. The mixture was stirred at rt for 30 min whereafter the solvents were evaporated under reduced pressure. Half of the amount of the acid chloride (1040 mg, 2.20 mmol) suspended in DCM (250 ml) was added dropwise during 31 h to a mixture of (1R,2S)-cyclohexane-1,2-diamine (5000 mg, 43.79 mmol), 1M NaOH (aq) (50 ml) and DCM (50 ml). After the addition was complete water was added and the phases were separated. The organic phase was washed with HCl (10%, aq) and brine, dried (MgSO₄), filtered and evaporated yield the product (crude 1312 mg)

¹H NMR (400 MHz, CDCl₃) δ 8.57 (2H, br), 7.69 (1H, br), 7.37-6.90 (12H, m), 5.00 (2H, s), 4.41 (1H, br), 3.72 (1H, br), 2.42 (3H, s), 2.18-1.40 (8H, m).

MS m/z 549 (M+H)⁺.

### Step 2 N-[(1S,2R)-2-aminocyclohexyl]-2-(2,4-dichlorophenyl)-1-(4-hydroxyphenyl)-5-methyl-1H-imidazole-4-carboxamide

N-[(1S,2R)-2-aminocyclohexyl]-1-[4-(benzyloxy)phenyl]-2-(2,4-dichlorophenyl)-5-methyl-1H-imidazole-4-carboxamide (791 mg, 1.44 mmol) was suspended in DCM (5 ml) and dimethyl sulfide (894 mg, 14.39 mmol) was added followed by boron trifluoride (2043 mg, 14.39 mmol). The reaction mixture was stirred at rt for 2.5 days (dark). Water and EtOAc were added and the phases separated. The organic phase was dried (MgSO₄), filtered and evaporated to yield the product (crude 715 mg).

MS m/z 459 (M+H)⁺.

### Step 3 N-[(1S,2R)-2-aminocyclohexyl]-1-(4-{[tert-butyl(dimethyl)silyl]oxy}phenyl)-2-(2,4-dichlorophenyl)-5-methyl-1H-imidazole-4-carboxamide

N-[(1S,2R)-2-aminocyclohexyl]-2-(2,4-dichlorophenyl)-1-(4-hydroxyphenyl)-5-methyl-1H-imidazole-4-carboxamide (715 mg, 1.56 mmol) was suspended in DCM (15 ml) and TEA (987 mg, 9.76 mmol) was added followed by TBDMSCl (985 mg, 6.53 mmol). The reaction mixture was stirred at rt for 22 h. DCM and water were added and the phases separated. The organic phase was dried (MgSO₄), filtered and evaporated to yield the product as an oil (crude 1140 mg).

MS m/z 573 (M+H)⁺.

### Step 4 tert-butyl [(1R,2S)-2-({[1-(4-{[tert-butyl(dimethyl silyl]oxy}phenyl-2-(2,4-dichlorophenyl)-5-methyl-1H-imidazol-4-yl]carbonyl}amino)cyclohexyl]carbamate

N-[(1S,2R)-2-aminocyclohexyl]-1-(4-{[tert-butyl(dimethyl)silyl]oxy}phenyl)-2-(2,4-dichlorophenyl)-5-methyl-1H-imidazole-4-carboxamide (1140 mg, 1.99 mmol) was suspended in THF (10 ml) and (Boc)₂O (444 mg, 2.03 mmol) was added. The reaction mixture was stirred at rt for 4 h whereafter the solvent was evaporated at reduced pressure and the residue redissolved in DCM. The organic phase was washed with water, dried (MgSO₄), filtered and evaporated. The residue was purified by flash chromatography (heptane:EtOAc; 9:1 then 0:10) to yield the product (620 mg, yield over 4 steps 70%).

¹H NMR (400 MHz, CDCl₃) δ 7.27 (1H, m), 7.11-6.98 (3H, m), 6.75-6.58 (4H, m), 5.01 (1H, br), 4.16 (1H, br), 3.68 (1H, br), 2.28 (3H, s), 1.63-1.34 (8H, m), 1.22 (9H, s), 0.77 (9H, s), 0.00 (6H, s). MS m/z 673 (M+H)⁺.

### Step 5 tert-butyl [(1R,2S-2-({[2-(2,4-dichlorophenyl)-1-(4-hydroxyphenyl)-5-methyl-1H imidazol-4-yl]carbonyl}amino)cyclohexyl]carbamate

tert-butyl [(1R,2S)-2-({[1-(4-{[tert-butyl(dimethyl)silyl]oxy}phenyl)-2-(2,4-dichlorophenyl)-5-methyl-1H-imidazol-4-yl]carbonyl}amino)cyclohexyl]carbamate (610 mg, 0.91 mmol) was suspended in dry THF (3 ml) and TBAF (1.0 M in THF) (237 mg, 0.91 mmol) was added. The reaction mixture was stirred at rt for 1 h 45 min. The solvent was evaporated and the residue redissolved in DCM, washed with water, dried (MgSO₄), filtered and evaporated. The residue was redissolved in EtOAc and some silica gel was added. The suspension was filtered through a plug of silica gel and eluted with EtOAc. The solvent was evaporated to yield the product (crude 529 mg).

¹H NMR (400 MHz, CDCl₃) δ 7.35 (1H, m), 7.21-7.08 (3H, m), 6.81-6.67 (4H, m), 5.06 (1H, br), 4.23 (1H, br), 3.80 (1H, br), 2.32 (3H, s), 1.69-1.33 (8H, m), 1.31 (9H, s).

MS m/z 559 (M+H)⁺.

### Step 6 4-[4-[({(1S,2R)-2-[(tert-butoxycarbonyl)amino]cyclohexyl}amino)carbonyl]-2-(2,4-dichlorophenyl)-5-methyl-1H-imidazol-1-yl]phenyl 3,3,3-trifluoropropane-1-sulfonate

tert-butyl [(1R,2S)-2-({[2-(2,4-dichlorophenyl)-1-(4-hydroxyphenyl)-5-methyl-1H-imidazol-4-yl]carbonyl}amino)cyclohexyl]carbamate (506 mg, 0.91 mmol) was suspended in dry DCM (6 ml) and TEA (110 mg, 1.09 mmol) was added at rt. The resulting mixture was cooled to −78° C. and 3,3,3-trifluoro-propane-1-sulfonyl chloride (181 mg, 0.92 mmol) in 0.2 ml dry DCM was added dropwise. Stirred at −78° C. for 3 h adding more of 3,3,3-trifluoro-propane-1-sulfonyl chloride (2×43 mg, 0.22 mmol)) after 1.5 h and 2.5 h. The reaction mixture was washed with water and evaporated to yield the product (crude 655 mg). MS m/z 719 (M+H)⁺.

### Step 7 4-[4-({[(1S,2R)-2-aminocyclohexyl]amino}carbonyl)-2-(2,4-dichlorophenyl)-5-methyl-1H-imidazol-1-yl]phenyl 3,3,3-trifluoropropane-1-sulfonate

To a suspension of 4-[4-[({(1S,2R)-2-[(tert-butoxycarbonyl)amino]cyclohexyl}-amino)carbonyl]-2-(2,4-dichlorophenyl)-5-methyl-1H-imidazol-1-yl]phenyl 3,3,3-trifluoropropane-1-sulfonate (655 mg, 0.91 mmol) in MeOH (10 ml) at 0° C. was added dropwise a solution of thionyl chloride in MeOH (prepared by dropwise addition of thionyl chloride (5414 mg, 45.51 mmol) to MeOH (10 ml) at −40° C.). After the addition the ice bath was removed. The reaction mixture was stirred at rt for 1 h whereafter the solvents were evaporated. The product was purified by HPLC (30=>100% ACN (with 0.1% formic acid) in 0.1% formic acid (aq) during 40 min). The ACN was evaporated and the resulting mixture extracted with DCM. The organic phase was washed with NaHCO₃ (1 M), dried (MgSO₄), filtered and evaporated to yield the product as a slightly yellow solid (315 mg yield over 3 steps 56%).

¹H NMR (400 MHz, CDCl₃) δ 7.51 (1H, m), 7.32-7.13 (7H, m), 4.14 (1H, br), 3.50-3.46 (2H, m), 3.11 (1H, br), 2.81-2.74 (2H, m), 2.47 (3H, s), 1.71-1.44 (8H, m).

HRMS Calcd for [C₂₆H₂₇Cl₂F₃N₄O₄S+H]⁺: 619.116. Found: 667.122.

### Example 16

### 4-[2-(2,4-dichlorophenyl)-4-({[(1S,2R)-2-(dimethylamino)cyclohexyl]amino}carbonyl)-5-methyl-1H-imidazol-1-yl]phenyl 3,3,3-trifluoropropane-1-sulfonate

### Step 1 1-[4-(benzyloxy)phenyl]-2-(2,4-dichlorophenyl)-N-[(1S,2R)-2-(dimethylamino)cyclohexyl]-5-methyl-1H-imidazole-4-carboxamide

To a suspension of N-[(1S,2R)-2-aminocyclohexyl]-1-[4-(benzyloxy)phenyl]-2-(2,4-dichlorophenyl)-5-methyl-1H-imidazole-4-carboxamide, prepared as in Ex. 15, Step 1 (493 mg, 0.90 mmol) in ACN (10 ml) was added formaldehyde, 36% (135 mg, 4.49 mmol) and sodium borohydride (75 mg, 1.97 mmol) in portions. The suspension was stirred at rt for 2 days adding after 2.5 h sodium borohydride (77 mg, 2.04 mmol), 3.5 h formaldehyde, 36% (67 mg, 2.24 mmol), 18.5 h formaldehyde, 36% (67 mg, 2.24 mmol) and sodium borohydride (77 mg, 2.04 mmol) (the temperature was increased to 40° C. for 4.5 h), 23 h acetic acid (1.85 ml) at rt, 28 h formaldehyde, 36% (135 mg, 4.49 mmol) followed by sodium cyano borohydride (112 mg, 1.78 mmol), 42 h formaldehyde, 36% (135 mg, 4.49 mmol) followed by sodium cyano borohydride (126 mg, 2.01 mmol). The reaction mixture was diluted with DCM, washed with 1M NaOH (aq) and brine, dried (MgSO₄), filtered and evaporated. The residue was purified by HPLC (30=>100% ACN in 0.1 M ammonium acetate during 30 min). The ACN was evaporated and the resulting mixture extracted with DCM, dried (MgSO₄), filtered and evaporated to yield the product (163 mg, 32%).

MS m/z 577 (M+H)⁺.

### Step 2 2-(2,4-dichlorophenyl)-N-[(1S,2R)-2-(dimethylamino)cyclohexyl]-1-(4-hydroxyphenyl)-5-methyl-1H-imidazole-4-carboxamide

1-[4-(benzyloxy)phenyl]-2-(2,4-dichlorophenyl)-N-[(1S,2R)-2-(dimethylamino)cyclohexyl]-5-methyl-1H-imidazole-4-carboxamide (163 mg, 0.28 mmol) was suspended in DCM (2 ml) and dimethyl sulfide (351 mg, 5.64 mmol) was added followed by boron trifluoride (801 mg, 5.64 mmol). The reaction mixture was stirred at rt for 2 days (dark) adding more of dimethyl sulfide (176 mg, 2.82 mmol) and boron trifluoride (401 mg, 2.82 mmol) after 17 h. Water and DCM were added and the phases separated. The organic phase was washed with water, dried (MgSO₄), filtered and evaporated to yield the product (crude 104 mg).

MS m/z 487 (M+H)⁺.

### Step 3 4-[2-(2,4-dichlorophenyl)-4-({[(1S,2R)-2-(dimethylamino)cyclohexyl]amino}-carbonyl)-5-methyl-1H-imidazol-1-yl]phenyl 3,3,3-trifluoropropane-1-sulfonate

2-(2,4-dichlorophenyl)-N-[(1S,2R)-2-(dimethylamino)cyclohexyl]-1-(4-hydroxyphenyl)-5-methyl-1H-imidazole-4-carboxamide (104 mg, 0.21 mmol) was suspended in dry DCM (1.5 ml) and TEA (26 mg, 0.26 mmol) was added at rt. The resulting mixture was cooled to −78° C. and 3,3,3-trifluoro-propane-1-sulfonyl chloride (50 mg, 0.26 mmol) in 0.5 ml dry DCM was added dropwise. Stirred at −78° C. for 6.5 h adding more of 3,3,3-trifluoro-propane-1-sulfonyl chloride (2×50 mg, 0.26 mmol)) after 2 h and 4 h and TEA (26 mg, 0.26 mmol) after 4 h. The reaction mixture was washed with water and evaporated. The residue was purified by HPLC (30=>100% ACN (with 0.1% formic acid) in 0.1% formic acid (aq) during 40 min) and freeze dried. The product was dissolved in DCM and washed with NaHCO₃ (1 M) and water, dried (MgSO₄), filtered and evaporated to yield the product as a slightly yellow oil (37 mg yield over 2 steps 20%).

¹H NMR (400 MHz, CDCl₃) δ 7.54 (1H, m), 7.38-7.16 (7H, m), 4.56 (1H, br), 3.54-3.51 (2H, m), 2.84-2.79 (2H, m), 2.51 (3H, s), 2.31 (6H, s), 2.22 (1H, m), 2.09-1.98 (2H, m), 1.82 (1H, m), 1.52-1.27 (5H, m). HRMS Calcd for [C₂₈H₃₁Cl₂F₃N₄O₄S+H]⁺: 647.147. Found: 647.147.

### Example 17

### 2-(2,4-dichlorophenyl)-5-methyl-N-piperidin-1-yl-1-[4-(3,3,3-trifluoropropoxy)phenyl]-1H-imidazole-4-carboxamide

It was prepared by using piperidin-1-amine hydrochloride as described in Ex 11, Step 5 to give 2-(2,4-dichlorophenyl)-5-methyl-N-piperidin-1-yl-1-[4-(3,3,3-trifluoropropoxy)-phenyl]-1H-imidazole-4-carboxamide (45 mg, 83 μmol, 41%) as a colorless solid.

¹H NMR (500 MHz, CDCl₃) δ 7.90 (s, 1H), 7.35-7.22 (m, 3H), 7.04 (d, J=8.7 Hz, 2H), 6.86 (d, J=8.7 Hz, 2H), 4.21-4.17 (m, 2H), 2.90-2.86 (m, 4H), 2.68-2.60 (m, 2H), 2.47 (s, 3H), 1.80-1.75 (m, 4H), 1.47-1.43 (m, 2H)

HRMS Calcd for [C₂₅H₂₅Cl₂F₃N₄O₂+H]⁺: 541.1385. Found: 541.1366.

new

### Step 1 Ethyl 2-(2,4-dichlorophenyl)-1-(4-hydroxyphenyl)-5-methyl-1H-imidazole-4-carboxylate

Ethyl 1-[4-(benzyloxy)phenyl]-2-(2,4-dichlorophenyl)-5-methyl-1H-imidazole-4-carboxylate, prepared as in Ex. 1, Step 2 (4.82 g, 10 mmol) was dissolved in 80 ml HBr (33% in acetic acid) and stirred overnight at room temperature with exclusion of light. The solvents were evaporated and the residue coevaporated with ethanol. The residue was dissolved in ethanol, 5 ml of HCl in dioxane (4M) and MgSO4 was added and the resulting mixture heated under reflux for 2.5 h. The reaction mixture was cooled to room temperature, filtered and evaporated. The residue was dissolved in EtOAc and washed with water basified with triethylamine and then brine. The organic layer was dried over Na₂SO₄ and evaporated to give ethyl 2-(2,4-dichlorophenyl)-1-(4-hydroxyphenyl)-5-methyl-1H-imidazole-4-carboxylate (4.74 g) as a brown, viscous oil of sufficient purity for the next step. ¹H NMR (500 MHz, CDCl₃) δ 8.65 (br. s, 1H), 7.28-7.14 (m, 3H), 6.88 (d, J=8.6 Hz, 2H), 6.79 (d, J=8.6 Hz, 2H), 4.32 (q, J=7.1 Hz, 2H), 2.43 (s, 3H), 1.27 (t, J=7.1 Hz, 3H)

### Step 2 Ethyl 2-(2,4-dichlorophenyl)-5-methyl-1-[4-(3,3,3-trifluoropropoxy)phenyl]-1H-imidazole-4-carboxylate

Ethyl 2-(2,4-dichlorophenyl)-1-(4-hydroxyphenyl)-5-methyl-1H-imidazole-4-carboxylate (978 mg, 2.5 mmol), 3,3,3-trifluoro-1-propanol (428 mg, 3.75 mmol) and triphenylphosphine (984 mg, 3.75 mmol) were dissolved in anhydrous THF (12 ml). Then DEAD (1.72 ml of a ca. 40% solution in toluene, d=0.95, 3.75 mmol) was added. The resulting mixture warms and was stirred at room temperature for 30 h, then heated to 50 C overnight. After cooling to room temperature, additional 3,3,3-trifluoro-1-propanol (428 mg, 3.75 mmol) and triphenylphosphine (984 mg, 3.75 mmol) followed by di-tert-butyl azodicarboxylate (863 mg, 3.75 mmol) was added and the resulting mixture stirred at room temperature overnight. Again, additional 3,3,3-trifluoro-1-propanol (428 mg, 3.75 mmol) and triphenylphosphine (984 mg, 3.75 mmol) followed by di-tert-butyl azodicarboxylate (863 mg, 3.75 mmol) was added and the resulting mixture stirred at room temperature overnight. The solvents were evaporated, the residue purified by column chromatography (silica gel, hexanes/EtOAc, 10-50%) to yield ethyl 2-(2,4-dichlorophenyl)-5-methyl-1-[4-(3,3,3-trifluoropropoxy)phenyl]-1H-imidazole-4-carboxylate (880 mg, 1.72 mmol, 68%) as a yellowish foam of sufficient purity for the next transformation.

¹H NMR (500 MHz, CDCl₃) δ 7.22-7.16 (m, 3H), 7.01 (d, J=8.7 Hz, 2H), 6.83 (d, J=8.7 Hz, 2H), 4.40 (q, J=7.1 Hz, 2H), 4.22-4.10 (m, 2H), 2.66-2.54 (m, 2H), 2.40 (s, 3H), 1.40 (t, J=7.1 Hz, 3H)

### Step 3 2-(2,4-dichlorophenyl)-5-methyl-1-[4-(3,3,3-trifluoropropoxy)phenyl]-1H-imidazole-4-carboxylic acid

Ethyl 2-(2,4-dichlorophenyl)-5-methyl-1-[4-(3,3,3-trifluoropropoxy)phenyl]-1H-imidazole-4-carboxylate (880 mg, 1.72 mmol) was dissolved in a mixture of 15 ml THF and 15 ml EtOH, then KOH (1.07 g, 19 mmol) dissolved in 10 ml water was added and the resulting mixture stirred at 50 C. After 3 h 30 min the reaction mixture was cooled to room temperature and the solvents were evaporated. The residue was partitioned between DCM and 1N HCl and after phase separation the aqueous layer was extracted two more times with DCM. The combined organic layers were dried over MgSO₄ and evaporated to give 2-(2,4-dichlorophenyl)-5-methyl-1-[4-(3,3,3-trifluoropropoxy)phenyl]-1H-imidazole-4-carboxylic acid (714 mg, 1.55 mmol, 90%) as a yellowish foam. ¹H NMR (500 MHz, CDCl₃) δ 7.32-7.18 (m, 3H), 7.00 (d, J=8.7 Hz, 2H), 6.85 (d, J=8.7 Hz, 2H), 4.18-4.14 (m, 2H), 2.66-2.55 (m, 2H), 2.42 (s, 3H)

### Step 3 2-(2,4-dichlorophenyl)-5-methyl-1-[4-(3,3,3-trifluoropropoxy)phenyl]-1H-imidazole-4-carbonyl chloride

2-(2,4-dichlorophenyl)-5-methyl-1-[4-(3,3,3-trifluoropropoxy)phenyl]-1H-imidazole-4-carboxylic acid (643 mg, 1.4 mmol) was dissolved in 10 ml DCM, then oxalyl chloride

(200 μl, 2.36 mmol) was added, followed by 10 μl DMF. The resulting mixture was stirred for 90 min at room temperature, then the solvents were evaporated and the residue dried in oil pump vacuum to give 2-(2,4-dichlorophenyl)-5-methyl-1-[4-(3,3,3-trifluoropropoxy)phenyl]-1H-imidazole-4-carbonyl chloride (690 mg, 1.37 mmol, 98%) as a yellowish foam which was used without further purification in the next step.

### Step 4 2-(2,4-dichlorophenyl)-5-methyl-N-piperidin-1-yl-1-[4-(3,3,3-trifluoropropoxy)phenyl]-1H-imidazole-4-carboxamide

To a mixture of piperidin-1-amine hydrochloride (0.3 mmol) and 100 pt pyridine in 1 ml DCM was added crude 2-(2,4-dichlorophenyl)-5-methyl-1-[4-(3,3,3-trifluoropropoxy)phenyl]-1H-imidazole-4-carbonyl chloride (96 mg, 0.2 mmol) in 1 ml DCM and the resulting mixture stirred at room temperature for 2 h 30 min. The reaction mixture was washed with 2 ml of sat. NaHCO₃ and after phase separation filtered through a phase separator. The solvents were evaporated and the residue purified by preparative HPLC eluting on a reverse-phase column with 5 to 100% acetonitrile in 0.1 M NH₄Ac to give 2-(2,4-dichlorophenyl)-5-methyl-N-piperidin-1-yl-1-[4-(3,3,3-trifluoropropoxy)phenyl]-1H-imidazole-4-carboxamide (45 mg, 83 μmol, 41%) as a colorless solid. ¹H NMR (500 MHz, CDCl₃) δ 7.90 (s, 1H), 7.35-7.22 (m, 3H), 7.04 (d, J=8.7 Hz, 2H), 6.86 (d, J=8.7 Hz, 2H), 4.21-4.17 (m, 2H), 2.90-2.86 (m, 4H), 2.68-2.60 (m, 2H), 2.47 (s, 3H), 1.80-1.75 (m, 4H), 1.47-1.43 (m, 2H). HRMS Calcd for [C₂₅H₂₅Cl₂F₃N₄O₂+H]⁺: 541.1385. Found: 541.1366.

### Example 18

### N-cyclohexyl-2-(2,4-dichlorophenyl)-5-methyl-1-[4-(3,3,3-trifluoropropoxy)phenyl]-1H-imidazole-4-carboxamide

It was prepared by using cyclohexylamine as described in Ex 11, Step 5 to give N-cyclohexyl-2-(2,4-dichlorophenyl)-5-methyl-1-[4-(3,3,3-trifluoropropoxy)phenyl]-1H-imidazole-4-carboxamide (53 mg, 98 μmol, 49%) as a colorless solid.

¹H NMR (500 MHz, CDCl₃) δ 7.35-7.22 (m, 3H), 7.11 (d, J=8.4 Hz, 1H), 7.04 (d, J=8.7 Hz, 2H), 6.87 (d, J=8.7 Hz, 2H), 4.21-4.17 (m, 2H), 4.00-3.93 (m, 1H), 2.69-2.60 (m, 2H), 2.48 (s, 3H), 2.05-2.02 (m, 2H), 1.80-1.76 (m, 2H), 1.68-1.64 (m, 1H), 1.47-1.38 (m, 2H), 1.36-1.16 (m, 3H). HRMS Calcd for [C₂₆H₂₆Cl₂F₃N₃O₂+H]⁺: 540.1432. Found: 540.1409.

### Example 19

### 2-(2,4-dichlorophenyl)-N-(4,4-difluorocyclohexyl)-5-methyl-1-[4-(3,3,3-trifluoropropoxy)phenyl]-1H-imidazole-4-carboxamide

It was prepared by using 4,4-difluorocyclohexylamine as described in Ex 11, Step 5 to give 2-(2,4-dichlorophenyl)-N-(4,4-difluorocyclohexyl)-5-methyl-1-[4-(3,3,3-trifluoropropoxy)phenyl]-1H-imidazole-4-carboxamide (65 mg, 112 μmol, 56%) as a colorless solid.

¹H NMR (500 MHz, CDCl₃) δ 7.36-7.22 (m, 3H), 7.15 (d, J=8.2 Hz, 1H), 7.05 (d, J=8.7 Hz, 2H), 6.87 (d, J=8.7 Hz, 2H), 4.21-4.18 (m, 2H), 4.11-4.09 (m, 1H), 2.69-2.60 (m, 2H), 2.48 (s, 3H), 2.16-2.09 (m, 4H), 1.98-1.86 (m, 2H), 1.73-1.66 (m, 2H)

HRMS Calcd for [C₂₆H₂₄Cl₂F₅N₃O₂+H]⁺: 576.1244. Found: 576.1238.

### Example 20

### 2-(2,4-dichlorophenyl)-5-methyl-N-(5-methylpyridin-2-yl)-1-[4-(3,3,3-trifluoropropoxy)phenyl]-1H-imidazole-4-carboxamide

It was prepared by using 2-amino-5-picoline as described in Ex 11, Step 5 to give 2-(2,4-dichlorophenyl)-5-methyl-N-(5-methylpyridin-2-yl)-1-[4-(3,3,3-trifluoropropoxy)phenyl]-1H-imidazole-4-carboxamide (30 mg, 54 μmol, 27%) as a colorless solid.

¹H NMR (500 MHz, CDCl₃) δ 9.68 (s, 1H), 8.27 (d, J=8.3 Hz, 1H), 8.15 (s, 1H), 7.56-7.53 (m, 1H), 7.36-7.23 (m, 3H), 7.07 (d, J=8.7 Hz, 2H), 6.89 (d, J=8.7 Hz, 2H), 4.22-4.19 (m, 2H), 2.70-2.61 (m, 2H), 2.53 (s, 3H), 2.32 (s, 3H)

HRMS Calcd for [C₂₆H₂₁Cl₂F₃N₄O₂+H]⁺: 549.1072. Found: 549.1080.

### Example 21

### 2-(2,4-dichlorophenyl)-1-[4-(3-fluoropropoxy)phenyl]-5-methyl-N-piperidin-1-yl-1H-imidazole-4-carboxamide

### Step 1 Ethyl 2-(2,4-dichlorophenyl)-1-[4-(3-fluoropropoxy)phenyl]-5-methyl-1H-imidazole-4-carboxylate

Ethyl 2-(2,4-dichlorophenyl)-1-(4-hydroxyphenyl)-5-methyl-1H-imidazole-4-carboxylate, Ex. 11, Step 1 (978 mg, 2.5 mmol), 3-fluoropropane-1-ol (293 mg, 3.75 mmol) and triphenylphosphine (984 mg, 3.75 mmol) were dissolved in anhydrous THF (9 ml), then DEAD (1.72 ml of a ca. 40% solution in toluene, d=0.95, 3.75 mmol) was added. The resulting mixture warms and was stirred at room temperature overnight. The residue was purified by column chromatography (silica gel, hexanes/EtOAc, 20-40%). The product-containing fractions were combined and evaporated. The residue was dissolved in DCM, then an equal amount of hexane was added. The resulting solid was filtered off, the filtrate concentrated to yield ethyl 2-(2,4-dichlorophenyl)-1-[4-(3-fluoropropoxy)phenyl]-5-methyl-1H-imidazole-4-carboxylate (1.07 g, 2.14 mmol, 85%) as a colorless foam of ca. 90% purity which was used in the next transformation without further purification.

¹H NMR (500 MHz, CDCl₃) δ 7.35-7.20 (m, 3H), 7.03 (d, J=8.7 Hz, 2H), 6.87 (d, J=8.7 Hz, 2H), 4.73-4.60 (m, 2H), 4.44 (q, J=7.1 Hz, 2H), 4.11-4.07 (m, 2H), 2.44 (s, 3H), 2.24-2.13 (m, 2H), 1.44 (t, J=7.1 Hz, 3H)

### Step 2 2-(2,4-dichlorophenyl)-1-[4-(3-fluoropropoxy)phenyl]-5-methyl-1H-imidazole-4-carboxylic acid

Ethyl 2-(2,4-dichlorophenyl)-1-[4-(3-fluoropropoxy)phenyl]-5-methyl-1H-imidazole-4-carboxylate (1.07 g, 2.14 mmol, ca. 90% pure) was dissolved in a mixture of 20 ml THF and 20 ml EtOH, then KOH (1.40 g, 25 mmol) dissolved in 10 ml water was added and the resulting mixture stirred at 50 C. After 3 h 30 min the reaction mixture was cooled to room temperature and the solvents were evaporated. The residue was partitioned between DCM and 1N HCl and after phase separation the aqueous layer extracted with DCM and twice with EtOAc. The combined organic layers were dried over MgSO₄ and evaporated to give 2-(2,4-dichlorophenyl)-1-[4-(3-fluoropropoxy)phenyl]-5-methyl-1H-imidazole-4-carboxylic acid (856 mg, 1.82 mmol, 85%) as a yellowish foam which was sufficiently pure for the next step.

¹H NMR (500 MHz, CDCl₃) δ 7.35-7.22 (m, 3H), 7.04 (d, J=8.7 Hz, 2H), 6.88 (d, J=8.7 Hz, 2H), 4.72-4.60 (m, 2H), 4.12-4.09 (m, 2H), 2.46 (s, 3H), 2.25-2.14 (m, 2H)

### Step 3 2-(2,4-dichlorophenyl)-1-[4-(3-fluoropropoxy)phenyl]-5-methyl-1H-imidazole-4-carbonyl chloride

2-(2,4-dichlorophenyl)-1-[4-(3-fluoropropoxy)phenyl]-5-methyl-1H-imidazole-4-carboxylic acid (732 mg, 1.55 mmol) was dissolved in 20 ml DCM, then oxalyl chloride (200 μl, 2.36 mmol) was added, followed by 10 μl DMF. The resulting mixture was stirred for 90 min at room temperature, then the solvents were evaporated and the residue dried in oil pump vacuum to give 2-(2,4-dichlorophenyl)-1-[4-(3-fluoropropoxy)phenyl]-5-methyl-1H-imidazole-4-carbonyl chloride (680 mg, 1.55 mmol, 99%) as a yellowish foam which was used without further purification in the next step.

### Step 4 2-(2,4-dichlorophenyl)-1-[4-(3-fluoropropoxy)phenyl]-5-methyl-N-piperidin-1-yl-1H-imidazole-4-carboxamide

To a mixture of piperidin-1-amine hydrochloride (0.39 mmol) and 100 μl pyridine in 2 ml DCM was added crude 2-(2,4-dichlorophenyl)-1-[4-(3-fluoropropoxy)phenyl]-5-methyl-1H-imidazole-4-carbonyl chloride (115 mg, 0.26 mmol) in 2 ml DCM and the resulting mixture stirred at room temperature for 2 h. The reaction mixture was washed with 2 ml of sat. NaHCO₃ and after phase separation filtered through a phase separator. The solvents were evaporated and the residue purified by preparative HPLC eluting on a reverse-phase column with 5 to 100% acetonitrile in 0.1 M NH₄Ac to give 2-(2,4-dichlorophenyl)-1-[4-(3-fluoropropoxy)phenyl]-5-methyl-N-piperidin-1-yl-1H-imidazole-4-carboxamide (74 mg, 146 μmol, 56%) as a colorless solid.

¹H NMR (500 MHz, CDCl₃) δ 7.90 (s, 1H), 7.35-7.22 (m, 3H), 7.00 (d, J=8.7 Hz, 2H), 6.87 (d, J=8.7 Hz, 2H), 4.72-4.60 (m, 2H), 4.11-4.08 (m, 2H), 2.89-2.86 (m, 4H), 2.47 (s, 3H), 2.24-2.13 (m, 2H), 1.80-1.75 (m, 4H), 1.47-1.43 (m, 2H)

HRMS Calcd for [C₂₅H₂₇Cl₂FN₄O₂+H]⁺: 505.1573. Found: 505.1572.

### Example 22

### N-cyclohexyl-2-(2,4-dichlorophenyl)-1-[4-(3-fluoropropoxy)phenyl]-5-methyl-1H-imidazole-4-carboxamide

It was prepared as described in Ex. 21, Step 4 by using cyclohexylamine as amine component to yield N-cyclohexyl-2-(2,4-dichlorophenyl)-1-[4-(3-fluoropropoxy)phenyl]-5-methyl-1H-imidazole-4-carboxamide (43 mg, 85 μmol, 32%) as a colorless solid.

¹H NMR (500 MHz, CDCl₃) δ 7.35-7.21 (m, 3H), 7.11 (d, J=8.2 Hz, 1H), 7.02 (d, J=8.7 Hz, 2H), 6.86 (d, J=8.7 Hz, 2H), 4.72-4.60 (m, 2H), 4.11-4.08 (m, 2H), 4.00-3.93 (m, 1H), 2.48 (s, 3H), 2.35-2.13 (m, 2H), 2.06-2.02 (m, 2H), 1.80-1.76 (m, 2H), 1.68-1.65 (m, 1H), 1.47-1.38 (m, 2H), 1.32-1.17 (m, 3H). HRMS Calcd for [C₂₆H₂₈Cl₂FN₃O₂+H]⁺: 504.1621. Found: 504.1642.

### Example 23

### 2-(2,4-dichlorophenyl)-1-[4-(3-fluoropropoxy)phenyl]-N-[(cis)-2-hydroxycyclohexyl]-5-methyl-1H-imidazole-4-carboxamide

It was prepared as described in Ex. 21, Step 4 by using cis-2-aminocyclohexanol hydrochloride as amine component to yield 2-(2,4-dichlorophenyl)-1-[4-(3-fluoropropoxy)phenyl]-N-[(cis)-2-hydroxycyclohexyl]-5-methyl-1H-imidazole-4-carboxamide (58 mg, 111 μmol, 43%) as a colorless solid. ¹H NMR (500 MHz, CDCl₃) δ 7.46 (d, J=8.2 Hz, 1H), 7.35-7.22 (m, 3H), 7.02 (d, J=8.7 Hz, 2H), 6.86 (d, J=8.7 Hz, 2H), 4.72-4.60 (m, 2H), 4.20-4.16 (m, 1H), 4.11-4.08 (m, 2H), 4.06-4.03 (m, 1H), 2.78-2.76 (m, 1H), 2.47 (s, 3H), 2.13-2.24 (m, 2H), 1.85-1.66 (m, 6H), 1.49-1.42 (m, 2H)

HRMS Calcd for [C₂₆H₂₈Cl₂FN₃O₃+H]⁺: 520.1570. Found: 520.1573.

### Example 24

### 2-(2,4-dichlorophenyl)-N-(4,4-difluorocyclohexyl)-1-[4-(3-fluoropropoxy)phenyl]-5-methyl-1H-imidazole-4-carboxamide

It was prepared as described in Ex. 21, Step 4 by using 4,4-difluorocyclohexylamine as amine component to give 2-(2,4-dichlorophenyl)-N-(4,4-difluorocyclohexyl)-1-[4-(3-fluoropropoxy)phenyl]-5-methyl-1H-imidazole-4-carboxamide (59 mg, 109 μmol, 42%) as a colorless solid. ¹H NMR (500 MHz, CDCl₃) δ 7.35-7.22 (m, 3H), 7.14 (d, J=8.2 Hz, 1H), 7.02 (d, J=8.7 Hz, 2H), 6.86 (d, J=8.7 Hz, 2H), 4.72-4.60 (m, 2H), 4.11-4.08 (m, 3H), 2.48 (s, 3H), 2.24-2.09 (m, 6H), 1.99-1.86 (m, 2H), 1.73-1.65 (m, 2H)

HRMS Calcd for [C₂₆H₂₆Cl₂F₃N₃O₂+H]⁺: 540.1432. Found: 540.1416.

### Example 25

### 2-(2,4-dichlorophenyl)-1-[4-(3-fluoropropoxy)phenyl]-5-methyl-N-(5-methylpyridin-2-yl)-1H-imidazole-4-carboxamide

It was prepared as described in Ex. 21, Step 4 by using 2-amino-5-picoline as amine component to yield 2-(2,4-dichlorophenyl)-1-[4-(3-fluoropropoxy)phenyl]-5-methyl-N-(5-methylpyridin-2-yl)-1H-imidazole-4-carboxamide (16 mg, 31 μmol, 12%) as a colorless solid. ¹H NMR (500 MHz, CDCl₃) δ 9.68 (s, 1H), 8.28 (d, J=8.3 Hz, 1H), 8.14 (d, J=2.2 Hz, 1H), 7.56-7.53 (m, 1H), 7.35-7.23 (m, 3H), 7.04 (d, J=8.7 Hz, 2H), 6.89 (d, J=8.7 Hz, 2H), 4.73-4.60 (m, 2H), 4.12-4.09 (m, 2H), 2.53 (s, 3H), 2.32 (s, 3H), 2.25-2.14 (m, 2H)

HRMS Calcd for [C₂₆H₂₃Cl₂FN₄O₂+H]⁺: 513.1260. Found: 513.1260.

### Example 26

### 4-[2-(2,4-dichlorophenyl)-4-({[cis-3-hydroxycyclohexyl]amino}carbonyl)-5-methyl-1H-imidazol-1-yl]phenyl 3,3,3-trifluoropropane-1-sulfonate

### Step 1 1-[4-(benzyloxy)phenyl]-2-(2,4-dichlorophenyl)-N-(3-hydroxycyclohexyl)-5-methyl-1H-imidazole-4-carboxamide

1-[4-(benzyloxy)phenyl]-2-(2,4-dichlorophenyl)-5-methyl-1H-imidazole-4-carboxylic acid, prepared as in Ex. 1, Step 3 (2000 mg, 4.41 mmol) was suspended in DCM (50 ml) and oxalyl chloride (2800 mg, 22.06 mmol) was added at rt followed by one drop of DMF. The mixture was stirred at rt for 15 min whereafter the solvents were evaporated under reduced pressure. The acid chloride suspended in DCM (10 ml) was added dropwise to a mixture of 3-aminocyclohexanol (610 mg, 5.29 mmol), 1M NaOH (aq) (30 ml) and DCM (30 ml). Stirred at rt for 2 h, adding more of 3-aminocyclohexanol after 1 h 25 min (67 mg, 0.58 mmol) and 1 h 45 min (58 mg, 0.50 mmol). Water/DCM were added and the phases were separated. The organic phase was washed with HCl (10%, aq) and brine, dried (MgSO₄), filtered and evaporated yield the product (crude 2790 mg).

¹H NMR (400 MHz, CDCl₃) δ 7.40-7.16 (8H, m), 7.03-6.88 (4H, m), 5.01 (2H, s), 4.44-4.32 (0.5H, m), 4.18-4.11 (0.5H, m), 4.06-3.94 (0.5H, m), 3.76-3.66 (0.5H, m), 2.46 (3H, s), 2.03-1.10 (8H, m). MS m/z 550 (M+H)⁺.

### Step 2 2-(2,4-dichlorophenyl)-N-(3-hydroxycyclohexyl)-1-(4-hydroxyphenyl)-5-methyl-1H-imidazole-4-carboxamide

1-[4-(benzyloxy)phenyl]-2-(2,4-dichlorophenyl)-N-(3-hydroxycyclohexyl)-5-methyl-1H-imidazole-4-carboxamide (2790 mg, 5.07 mmol) was suspended in DCM (50 ml) and dimethyl sulfide (3149 mg, 50.68 mmol) was added followed by boron trifluoride diethyl etherate (5771 mg, 50.68 mmol). The reaction mixture was stirred at rt for 36 h (dark) adding more of dimethyl sulfide (3149 mg, 50.68 mmol) and boron trifluoride (5771 mg, 50.68 mmol) after 16 h. The solvent was evaporated and the residue redissolved in EtOAc/water. The phases were separated and the organic phase dried (MgSO₄), filtered and evaporated to yield the product (crude 2540 mg). MS m/z 460 (M+H)⁺.

### Step 3 4-[2-(2,4-dichlorophenyl)-4-({[cis-3-hydroxycyclohexyl]amino}carbonyl)-5-methyl-1H-imidazol-1-yl]phenyl 3,3,3-trifluoropropane-1-sulfonate

2-(2,4-dichlorophenyl)-N-(3-hydroxycyclohexyl)-1-(4-hydroxyphenyl)-5-methyl-1H-imidazole-4-carboxamide (2527 mg, 5.49 mmol) was suspended in dry DCM (20 ml) and TEA (667 mg, 6.59 mmol) was added at rt. The resulting mixture was cooled to −78° C. and 3,3,3-trifluoro-propane-1-sulfonyl chloride (1295 mg, 6.59 mmol) was added dropwise. Stirred at −78° C. for 2 h 45 min. When the reaction mixture had reached rt it was washed with water and evaporated. The stereoisomers were separated by HPLC (30=>100% ACN in 0.1 M ammonium acetate) to yield the cis-hydroxycyclohexyl (152 mg, 5.6% over three steps). ¹H NMR (400 MHz, CDCl₃) δ 7.41-7.11 (7H, m), 4.08-3.94 (1H, m), 3.80-3.69 (1H, m), 3.53-3.45 (2H, m), 2.85-2.71 (2H, m), 2.48 (3H, s), 2.33-2.24 (1H, m), 1.99-1.88 (2H, m), 1.86-1.77 (1H, m), 1.44-1.15 (4H, m). HRMS Calcd for [C₂₆H₂₆Cl₂F₃N₃O₅S+H]⁺: 620.100. Found: 620.102.

### Example 27

### 4-[2-(2,4-dichlorophenyl)-4-({[trans-3-hydroxycyclohexyl]amino}carbonyl)-5-methyl-1H-imidazol-1-yl]phenyl 3,3,3-trifluoropropane-1-sulfonate

The title compound was obtained as described in Ex. 26, Step 3 (205 mg, 7.5% over three steps) as white solids. ¹H NMR (400 MHz, CDCl₃) δ 7.33-7.06 (7H, m), 4.43-4.32 (1H, m), 4.15-4.08 (1H, m), 3.52-3.45 (2H, m), 2.86-2.71 (2H, m), 2.47 (3H, s), 2.02-1.33 (8H, m). HRMS Calcd for [C₂₆H₂₆Cl₂F₃N₃O₅S+H]⁺: 620.100. Found: 620.103.

### Example 28

### 4-{2-(2-chlorophenyl)-4-[(cyclohexylamino)carbonyl]-5-methyl-1H-imidazol-1-yl}phenyl 3,3,3-trifluoropropane-1-sulfonate

### Step 1 N-[4-(benzyloxy)phenyl]-2-chlorobenzenecarboximidamide

[4-(benzyloxy)phenyl]amine hydrochloride (5000 mg, 21.21 mmol) was suspended in THF (25 ml, dry) under N₂ and the beige suspension was cooled to −10 C. Ethylmagnesium bromide (44.5 ml 1M in THF) was added and the cooling bath was removed after the addition. After 20 min, the reaction mixture was now clear, 2-chlorobenzonitrile (2923 mg, 21.25 mmol) dissolved in THF (25 ml, dry) was added dropwise during 10 min. The reaction mixture was stirred at rt o.n. Water (50 ml) was carefully added and the THF/water mixture extracted with ethyl acetate. The organic phase was dried (MgSO₄), filtered and evaporated to yield the product as a beige solid (crude 7287 mg).

¹H NMR (400 MHz, CDCl₃) δ 7.61-7.47 (1H, m), 7.44-7.22 (8H, m), 6.99-6.79 (4H, m), 5.00 (2H, s). MS m/z 337 (M+H)⁺.

### Step 2 Ethyl 1-[4-(benzyloxy)phenyl]-2-(2-chlorophenyl)-5-methyl-1H-imidazole-4-carboxylate

N-[4-(benzyloxy)phenyl]-2-chlorobenzenecarboximidamide (7263 mg, 21.56 mmol) was dissolved in THF (60 ml) and potassium carbonate (2980 mg, 21.56 mmol) was added. After having stirred the reaction mixture for 10 min ethyl 3-bromo-2-oxobutanoate (5449 mg, 26.07 mmol) was added dropwise during 1 h. The reaction mixture was stirred at rt over the weekend. A precipitate had formed and the suspension was light yellow. The white precipitate was filtered off and the filtrate was evaporated under reduced pressure. The residue was dissolved in acetic acid (60 ml) and refluxed for 1 h 15 min. The reaction mixture was left with stirring at rt o.n. Water was added and the product was extracted with EtOAc. The organic phase was washed with NaHCO₃ (sat), dried (MgSO₄), filtered and evaporated to yield the product (crude 6381 mg). MS m/z 447 (M+H)⁺.

### Step 3 1-[4-(benzyloxy)phenyl]-2-(2-chlorophenyl)-5-methyl-1H-imidazole-4-carboxylic acid

Ethyl 1-[4-(benzyloxy)phenyl]-2-(2-chlorophenyl)-5-methyl-1H-imidazole-4-carboxylate (6381 mg, 14.28 mmol) was dissolved in THF (60 ml) and LiOH (1368 mg, 57.11 mmol) dissolved in water (30 ml) was added. The reaction mixture was stirred at rt for 1 h whereafter NaOH (2284 mg, 57.11 mmol) dissolved in water (30 ml) was added. After totally 2 h at rt the THF was evaporated under reduced pressure and ethanol (90 ml) was added. The suspension was heated at 80° C. for 25 min whereafter the ethanol was evaporated under reduced pressure. Water was added and the mixture acidified with HCl (aq, 10%). The precipitate formed was collected by filtration, redissolved in DCM, dried (MgSO₄), filtered and evaporated to yield the product (crude 2380 mg). MS m/z 419 (M+H)⁺.

### Step 4 2,2,2-trichloroethyl 1-[4-(benzyloxy phenyl]-2-(2-chlorophenyl)-5-methyl-1H-imidazole-4-carboxylate

1-[4-(benzyloxy)phenyl]-2-(2-chlorophenyl)-5-methyl-1H-imidazole-4-carboxylic acid (2380 mg, 5.68 mmol) was dissolved in DCM (60 ml) and oxalyl chloride (3606 mg, 28.41 mmol) was added at rt followed by one drop of DMF which resulted in gas formation. The mixture was stirred at rt for 35 min whereafter the solvents were evaporated. The acid chloride was dissolved in DCM (20 ml) and beta-trichloroethanol (933 mg, 6.25 mmol) was added followed by DIPEA (881 mg, 6.82 mmol) and DMAP (69 mg, 0.57 mmol). The reaction mixture was stirred at rt for 3 h whereafter the reaction mixture was diluted with DCM and washed with water, dried (MgSO₄), filtered and evaporated to yield the product (crude 3357 mg). MS m/z 549 (M+H)⁺.

### Step 5 2,2,2-trichloroethyl 2-(2-chlorophenyl)-1-(4-hydroxyphenyl)-5-methyl-1H-imidazole-4-carboxylate

2,2,2-trichloroethyl 1-[4-(benzyloxy)phenyl]-2-(2-chlorophenyl)-5-methyl-1H-imidazole-4-carboxylate (3357 mg, 6.10 mmol) was dissolved in 33% HBr in acetic acid (50 ml) during 1 h because of a very sticky starting material. After having stirred at rt for an additional 15 min ethanol (ca 50 ml) was added to the reaction mixture and the solvents were evaporated at reduced pressure. The product was redissolved in MeOH and neutralised with NaHCO₃ (1M, aq). The solvent was evaporated and the mixture redissolved in DCM/brine. The phases were separated and the water phase extracted with DCM. The combined organic phase was washed with water, dried (MgSO₄), filtered and evaporated to yield the product (crude 1800 mg). ¹H NMR (400 MHz, CDCl₃) δ 7.29-7.06 (4H, m), 6.89-6.64 (4H, m), 4.95 (2H, s), 2.43 (3H, s). MS m/z 459 (M+H)⁺.

### Step 6 2,2,2-trichloroethyl 2-(2-chlorophenyl-5-methyl-1-(4-{[(3,3,3-trifluoropropyl)sulfonyl]oxy}phenyl)-1H-imidazole-4-carboxylate

2,2,2-trichloroethyl 2-(2-chlorophenyl)-1-(4-hydroxyphenyl)-5-methyl-1H-imidazole-4-carboxylate (921 mg, 2.00 mmol) was suspended in dry DCM (20 ml) and TEA (243 mg, 2.40 mmol) was added at rt. The resulting mixture was cooled to −78° C. and 3,3,3-trifluoro-propane-1-sulfonyl chloride (440 mg, 2.24 mmol) was added dropwise. The reaction mixture was stirred at −78° C. for 2 h. When the reaction mixture had reached rt water was added and the organic phase was separated on a phase separator and evaporated to yield the product (crude 1320 mg). MS m/z 619 (M+H)⁺.

### Step 7 2-(2-chlorophenyl)-5-methyl-1-(4-{[(3,3,3-trifluoropropyl)sulfonyl]oxy}phenyl)-1H-imidazole-4-carboxylic acid

2,2,2-trichloroethyl 2-(2-chlorophenyl)-5-methyl-1-(4-{[(3,3,3-trifluoropropyl)sulfonyl]oxy}phenyl)-1H-imidazole-4-carboxylate (1294 mg, 2.09 mmol) was dissolved in acetic acid (20 ml) and zinc dust (810 mg, 12.39 mmol) was added. The reaction mixture was stirred at rt for 4 h, adding more zinc dust (213 mg, 3.26 mmol) after 3 h. DCM was added and the mixture was filtered through celite and evaporated. The residue was redissolved in DCM and washed with 1M HCl (aq), dried (MgSO₄), filtered, evaporated and coevaporated twice with toluene to yield the product (crude 804 mg).

MS m/z 489 (M+H)⁺.

### Step 8 4-[4-(chlorocarbonyl)-2-(2-chlorophenyl)-5-methyl-1H-imidazol-1-yl]phenyl 3,3,3-trifluoropropane-1-sulfonate

2-(2-chlorophenyl)-5-methyl-1-(4-{[(3,3,3-trifluoropropyl)sulfonyl]oxy}phenyl)-1H-imidazole-4-carboxylic acid (809 mg, 1.65 mmol) was dissolved in DCM (25 ml) and oxalyl chloride (1050 mg, 8.27 mmol) was added at rt followed by one drop of DMF which resulted in gas formation. The reaction mixture was stirred at rt for 45 min whereafter the solvents were evaporated to yield the product (crude 840 mg).

### Step 9

### 4-{2-(2-chlorophenyl)-4-[(cyclohexylamino)carbonyl]-5-methyl-1H-imidazol-1-yl}phenyl 3,3,3-trifluoropropane-1-sulfonate

4-[4-(chlorocarbonyl)-2-(2-chlorophenyl)-5-methyl-1H-imidazol-1-yl]phenyl 3,3,3-trifluoropropane-1-sulfonate (279 mg, 0.55 mmol) suspended in DCM (5 ml) was added dropwise to a mixture of cyclohexylamine (70 mg, 0.71 mmol), 1M NaOH (10 ml) and DCM (5 ml). The reaction mixture was stirred at rt for 2 h whereafter water/DCM was added and the phases were separated. The organic phase was washed with HCl (0.1 M, aq) and evaporated. The product was purified by HPLC (30=>100% ACN in 0.1 M ammonium acetate) to yield the product as a white solid (107 mg, yield over 9 steps 5.2%). ¹H NMR (400 MHz, CDCl₃) δ 7.38-7.08 (8H, m), 3.99-3.87 (1H, m), 3.50-3.42 (2H, m), 2.83-2.69 (2H, m), 2.49 (3H, s), 2.04-1.96 (2H, m), 1.79-1.69 (2H, m), 1.66-1.58 (1H, m), 1.44-1.10 (5H, m). HRMS Calcd for [C₂₆H₂₇ClF₃N₃O₄S+H]⁺: 570.144. Found: 570.142.

### Example 29

### 4-[2-(2-chlorophenyl)-4-({[cis-2-hydroxycyclohexyl]amino}carbonyl)-5-methyl-1H-imidazol-1-yl]phenyl 3,3,3-trifluoropropane-1-sulfonate

4-[4-(chlorocarbonyl)-2-(2-chlorophenyl)-5-methyl-1H-imidazol-1-yl]phenyl 3,3,3-trifluoropropane-1-sulfonate, prepared as in Ex. 28, Step 8 (279 mg, 0.55 mmol) suspended in DCM (5 ml) was added dropwise to a mixture of cis-2-aminocyclohexanol hydrochloride (101 mg, 0.67 mmol), 1M NaOH (10 ml) and DCM (5 ml). The reaction mixture was stirred at rt for 2 h whereafter water/DCM was added and the phases were separated. The organic phase was washed with HCl (0.1 M, aq) and evaporated. The product was purified by HPLC (30=>100% ACN in 0.1 M ammonium acetate) to yield the product as a white solid (89 mg, yield over 9 steps 4.2%). ¹H NMR (400 MHz, CDCl₃) δ 7.48-7.13 (8H, m), 4.18-4.08 (1H, m), 4.04-3.99 (1H, m), 3.48-3.44 (2H, m), 2.83-2.68 (2H, m), 2.48 (3H, s), 1.82-1.32 (8H, m). HRMS Calcd for [C₂₆H₂₇ClF₃N₃O₅S+H]⁺:

586.139. Found: 586.137.

### Example 30

### 2-(2,4-dichlorophenyl)-1-[4-(3-fluoropropoxy)phenyl]-5-methyl-N-piperidin-1-yl-1H-imidazole-4-carboxamide

It was prepared as in Ex. 21, Step 4 by using piperidin-1-amine hydrochloride as amine component gave 2-(2,4-dichlorophenyl)-1-[4-(3-fluoropropoxy)phenyl]-5-methyl-N-piperidin-1-yl-1H-imidazole-4-carboxamide (74 mg, 146 μmol, 56%) as a colorless solid.

¹H NMR (500 MHz, CDCl₃) δ 7.90 (s, 1H), 7.35-7.22 (m, 3H), 7.00 (d, J=8.7 Hz, 2H), 6.87 (d, J=8.7 Hz, 2H), 4.72-4.60 (m, 2H), 4.11-4.08 (m, 2H), 2.89-2.86 (m, 4H), 2.47 (s, 3H), 2.24-2.13 (m, 2H), 1.80-1.75 (m, 4H), 1.47-1.43 (m, 2H). HRMS Calcd for [C₂₅H₂₇Cl₂FN₄O₂+H]⁺: 505.1573. Found: 505.1572.

### Example 31

### 2-(2,4-dichlorophenyl)-N-[(cis)-2-hydroxycyclohexyl]-5-methyl-1-[4-(3,3,3-trifluoropropoxy)phenyl]-1H-imidazole-4-carboxamide

It was prepared as in Ex. 17, Step 4 by using cis-2-aminocyclohexanol hydrochloride as amine component to yield 2-(2,4-dichlorophenyl)-N-[(cis)-2-hydroxycyclohexyl]-5-methyl-1-[4-(3,3,3-trifluoropropoxy)phenyl]-1H-imidazole-4-carboxamide (46 mg, 82 μmol, 41%) as a colorless solid. ¹H NMR (500 MHz, CDCl₃) δ 7.46 (d, J=8.2 Hz, 1H), 7.35-7.22 (m, 3H), 7.04 (d, J=8.7 Hz, 2H), 6.87 (d, J=8.7 Hz, 2H), 4.21-4.16 (m, 3H), 4.06-4.04 (m, 1H), 2.77 (s, 1H), 2.68-2.60 (m, 2H), 2.47 (s, 3H), 1.83-1.43 (m, 8H). HRMS Calcd for [C₂₆H₂₆Cl₂F₃N₃O₃+H]⁺: 556.1382. Found: 556.1413.

### Example 32

### 4-{2-(2,4-dichlorophenyl)-4-[(4-hydroxycyclohexyl)carbamoyl]-5-methyl-1H-imidazol-1-yl}phenyl 3,3,3-trifluoropropane-1-sulfonate

### Step 1

### 2,2,2-trichloroethyl 1-[4-(benzyloxy)phenyl]-2-(2,4-dichlorophenyl)-5-methyl-1H-imidazole-4-carboxylate

1-[4-(benzyloxy)phenyl]-2-(2,4-dichlorophenyl)-5-methyl-1H-imidazole-4-carboxylic acid, from Ex. 1, Step 3 (10.00 g, 22.06 mmol) was dissolved in DCM (210 ml) and oxalyl chloride (1 8.45 g, 145.36 mmol) was added followed by a few drops of DMF. The mixture was stirred at rt for 2 h whereafter the solvents were evaporated. The residue was dissolved in DCM (80 ml) and at 0 C 2,2,2-trichloroethanol (3.63 g, 24.27 mmol) was added followed by DIPEA (3.42 g, 26.47 mmol). The icebath was removed after the addition of DIPEA and the reaction mixture stirred at rt for 3 h, adding DMAP (279 mg, 2.28 mmol) after 1 h 40 min. The reaction mixture was diluted with DCM, washed with water, dried (MgSO₄), filtered and evaporated to yield the product (crude 14.87 g). ¹H NMR (400 MHz, CDCl₃) δ 7.40-7.14 (8H, m), 7.04-6.98 (2H, m), 6.94-6.88 (2H, m), 5.01 (4H, s), 2.45 (3H, s). MS m/z 583 (M+H)⁺.

### Step 2

### 2,2,2-trichloroethyl 2-(2,4-dichlorophenyl)-1-(4-hydroxyphenyl)-5-methyl-1H-imidazole-4-carboxylate

2,2,2-trichloroethyl 1-[4-(benzyloxy)phenyl]-2-(2,4-dichlorophenyl)-5-methyl-1H-imidazole-4-carboxylate (crude 14.77 g) was dissolved in 33% HBr in acetic acid (200 ml) which took 1 h 30 min because of a very sticky starting material. After having stirred at rt for an additional hour the reaction mixture was cooled to 0 C and ethanol was added. The mixture was stirred for 10 min before the solvents were evaporated. The residue was redissolved in MeOH and neutralised with NaHCO₃ (1M, aq). The solvent was evaporated and the mixture redissolved in DCM. The organic phase was washed with brine and water, dried (MgSO₄), filtered and evaporated to yield the product (10.36 g, 95% over two steps). ¹H NMR (400 MHz, CDCl₃) δ 8.63 (1H, br), 7.25-7.08 (3H, m), 6.86-6.68 (4H, m), 4.95 (2H, s), 2.43 (3H, s). MS m/z 493 (M+H)⁺.

### Step 3

### 2,2,2-trichloroethyl 2-(2,4-dichlorophenyl)-5-methyl-1-(4-{[(3,3,3-trifluoropropyl)sulfonyl]oxy}phenyl)Y—H-imidazole-4-carboxylate

2,2,2-trichloroethyl 2-(2,4-dichlorophenyl)-1-(4-hydroxyphenyl)-5-methyl-1H-imidazole-4-carboxylate (5.01 g, 10.13 mmol) was suspended in dry DCM (100 ml) under nitrogen and TEA (1.23 g, 12.16 mmol) was added at rt. The resulting mixture was cooled to −78 C and 3,3,3-trifluoropropane-1-sulfonyl chloride (2.19 g, 11.14 mmol) was added dropwise. The reaction mixture was stirred at −78 C for 3 h adding more of 3,3,3-trifluoropropane-1-sulfonyl chloride (0.28 g 1.43 mmol) after 2 h. Water was added and the phases were separated on a phase separator. The organic phase was evaporated to yield the product (6.43 g, 97%). ¹H NMR (400 MHz, CDCl₃) δ 7.37-7.15 (7H, m), 5.01 (2H, s), 3.53-3.45 (2H, m), 2.84-2.70 (2H, m), 2.48 (3H, s). MS m/z 653 (M+H)⁺.

### Step 4

### 2-(2,4-dichlorophenyl)-5-methyl-1-(4-{[(3,3,3-trifluoropropyl)sulfonyl]oxy}phenyl)-1H-imidazole-4-carboxylic acid

2,2,2-trichloroethyl 2-(2,4-dichlorophenyl)-5-methyl-1-(4-{[(3,3,3-trifluoropropyl)sulfonyl]oxy}phenyl)-1H-imidazole-4-carboxylate (6.43 g, 9.82 mmol) was dissolved in acetic acid (100 ml) and zinc dust (9.74 g, 148.91 mmol) was added. The reaction mixture was stirred at rt for 3 h whereafter it was filtered through celite and evaporated. The residue was redissolved in DCM and washed with 0.1M HCl (aq), dried, filtered and evaporated to yield the product (crude 5.28 g). MS m/z 523 (M+H)⁺.

### Step 5

### 4-{2-(2,4-dichlorophenyl)-4-[(4-hydroxycyclohexyl)carbamoyl]-5-methyl-1H-imidazol-1-yl}phenyl 3,3,3-trifluoropropane-1-sulfonate

2-(2,4-dichlorophenyl)-5-methyl-1-(4-{[(3,3,3-trifluoropropyl)sulfonyl]oxy}phenyl)-1H-imidazole-4-carboxylic acid (crude 528 mg) was dissolved in DCM (25 ml) and oxalyl chloride (641 mg, 5.00 mmol) was added. A precipitate was formed immediately after the addition and more DCM (15 ml) was added followed by a few drops of DMF. The reaction mixture was stirred at rt for 2 h whereafter more oxalyl chloride (641 mg, 5.00 mmol) was added. After another 10 min the solvents were evaporated. Half of the crude material was suspended in DCM (5 ml) and added dropwise to a mixture of 4-aminocyclohexanol (74 mg, 0.64 mmol), 1M NaOH (10 ml) and DCM (5 ml). The reaction mixture was stirred at rt for 2 h whereafter water/DCM were added and the phases separated. The organic phase was washed with HCl (0.1 M, aq) and evaporated. The product was purified by HPLC to yield the title compound as a white solid after freeze drying (164 mg, 54% over two steps). Note that the product is a mixture of cis- and transisomers.

¹H NMR (400 MHz, CDCl₃) δ 7.33-6.98 (7H, m), 4.07-3.86 (1.5H, m), 3.67-3.54 (0.5H, m), 3.52-3.44 (2H, m), 2.85-2.71 (2H, m), 2.47 (3H, s), 2.12-1.95 (2H, m), 1.82-1.66 (4H, m), 1.49-1.26 (2H, m).

HRMS Calcd for [C₂₆H₂₆Cl₂ F₃N₃O₅S+H]⁺: 620.100. Found: 620.100.

### Examples 33-45

The following compounds are prepared in a similar manner to those described above:
Ex. 33 3,3,3-Trifluoropropane-1-sulfonic acid 4-[2-(3-cyano-5-fluorophenyl)-4-(4,4-difluorocyclohexylcarbamoyl)-5-methylimidazol-1-yl]phenyl ester
Ex. 34 3,3,3-Trifluoropropane-1-sulfonic acid 4-[2-(3-cyanophenyl)-4-(1-hydroxymethyl-3-methylbutylcarbamoyl)-5-methylimidazol-1-yl]phenyl ester
Ex. 35 3,3,3-Trifluoropropane-1-sulfonic acid 4-[4-((2-aminocyclohexylcarbamoyl)-2-(3-cyano-5-fluorophenyl)-5-methylimidazol-1-yl]phenyl ester
Ex. 36 3,3,3-Trifluoropropane-1-sulfonic acid 4-[2-(3-cyano-5-fluorophenyl)-4-(3-dimethylaminocyclohexylcarbamoyl)-5-methylimidazol-1-yl]phenyl ester
Ex. 37 3,3,3-Trifluoropropane-1-sulfonic acid 4-[2-(3-cyano-5-fluorophenyl)-4-((1S,2R)-2-hydroxycyclohexylcarbamoyl)-5-methylimidazol-1-yl]phenyl ester
Ex. 38 3,3,3-Trifluoropropane-1-sulfonic acid 4-[2-(3-cyanophenyl)-4-(2-hydroxy-cyclohexylcarbamoyl)-5-methylimidazol-1-yl]phenyl ester
Ex. 39 3,3,3-Trifluoropropane-1-sulfonic acid 4-[2-(3-cyano-5-fluorophenyl)-4-(3-hydroxycyclohexylcarbamoyl)-5-methylimidazol-1-yl]phenyl ester
Ex. 40 3,3,3-Trifluoropropane-1-sulfonic acid 4-[2-(2-chlorophenyl)-4-(2-hydroxy-cyclohexylcarbamoyl)-5-methylimidazol-1-yl]phenyl ester
Ex. 41 3,3,3-Trifluoropropane-1-sulfonic acid 4-[2-(2-chlorophenyl)-4-(4,4-difluoro-cyclohexylcarbamoyl)-5-methylimidazol-1-yl]phenyl ester
Ex. 42 3,3,3-Trifluoropropane-1-sulfonic acid 4-[2-(4-chloro-2-methylphenyl)-5-methyl-4-(piperidin-1-ylcarbamoyl)imidazol-1-yl]phenyl ester
Ex. 43 2-(2,4-Dichlorophenyl)-5-methyl-1-[4-(3,3,3-trifluoropropoxy)phenyl]-1H-imidazole-4-carboxylic acid (3-hydroxycyclohexyl)amide
Ex. 44 3-Fluoropropane-1-sulfonic acid 4-[2-(2,4-dichlorophenyl)-4-((1S,2R)-2-hydroxy-cyclohexylcarbamoyl)-5-methylimidazol-1-yl]phenyl ester
Ex. 45 4,4,4-Trifluorobutane-1-sulfonic acid 4-[2-(3-cyano-5-fluorophenyl)-4-(1-ethyl-butylcarbamoyl)-5-methylimidazol-1-yl]phenyl ester

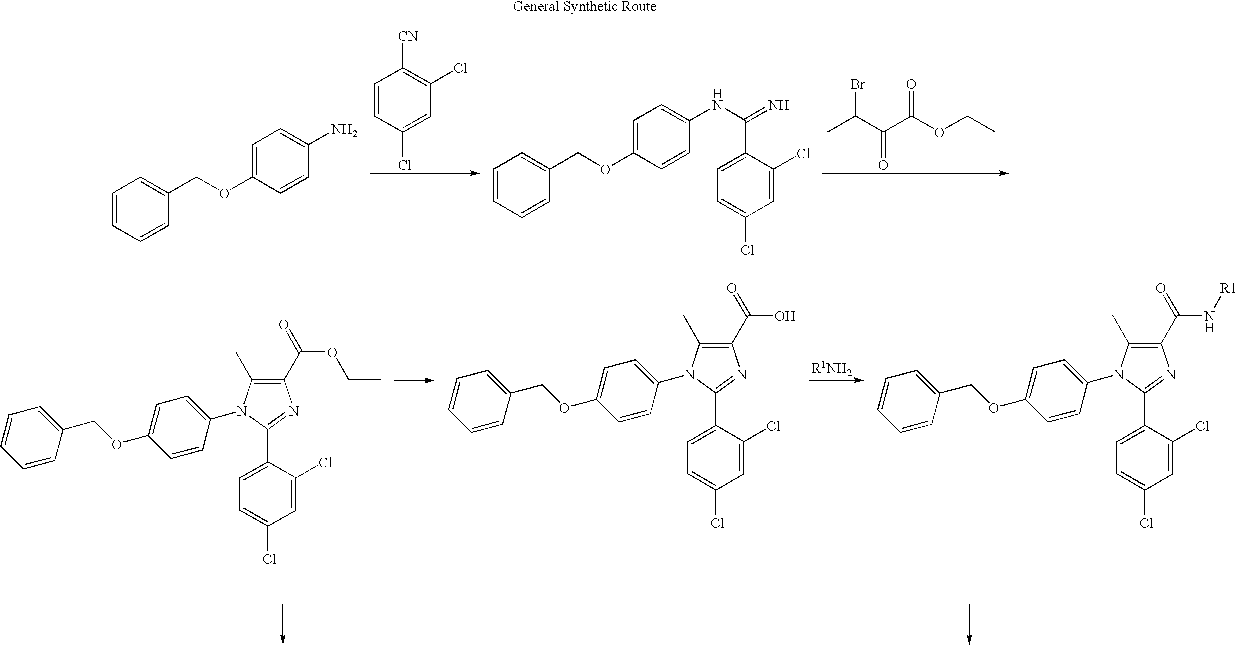

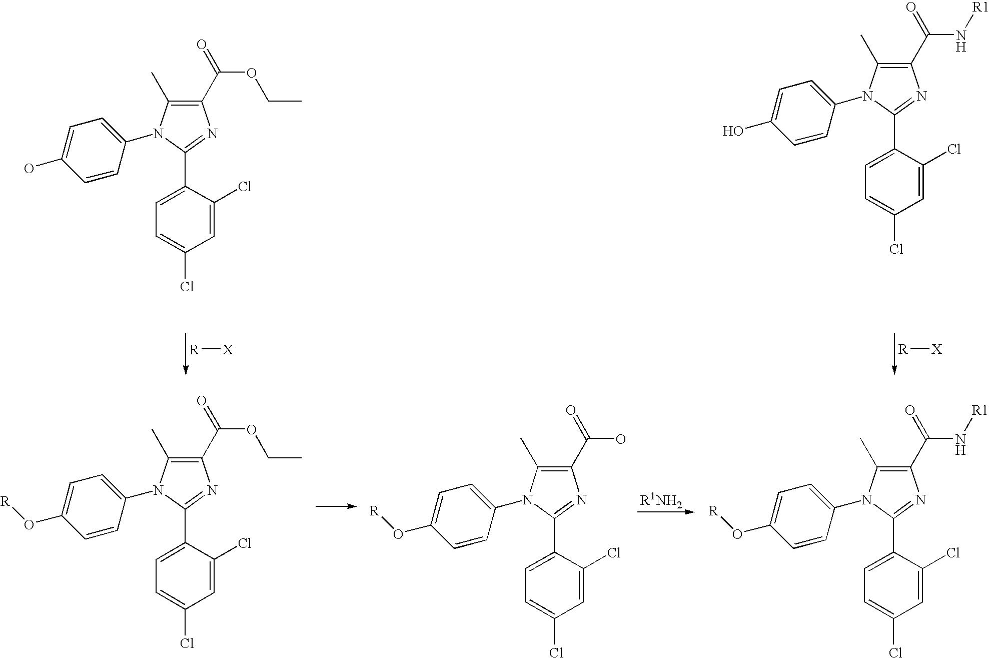

1: A compound of formula (I) 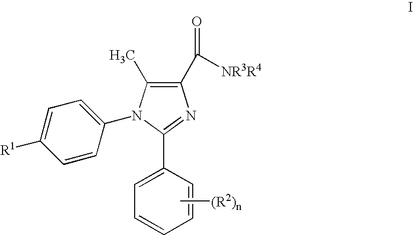
wherein
R¹ represents a group R⁵O— in which R⁵ represents a C₃₋₇alkyl group substituted by one or more fluoro or R⁵ represents a C₃₋₇alkylsulphonyl group which is substituted by one or more fluoro;
R² represents a C₁₋₄alkyl group, fluoro, chloro or cyano wherein each R² is independently selected when n is >1;
R³ represents H; and
R⁴ represents a) cyclohexyl optionally substituted by one or more of the following: hydroxy, fluoro, amino, mono or diC₁₋₃alkylamino b) piperidino substituted by one or more hydroxy c) unsubstituted piperidino but only when one of the following applies: R¹ represents 3-fluoropropylsulphonyloxy or 3,3,3-trifluoropropoxy or 3-fluoropropoxy or at least one R² represents methyl; d) phenyl substituted by one or more trifluoromethoxy e) pyridyl substituted by one or more of the following: a C₁₋₄alkyl group; trifluoromethyl; or fluoro; provided that R⁴ is not 5-trifluoromethyl-2-pyridyl or f) a C₄₋₉alkyl group optionally substituted by one or more hydroxy;
n is 1, 2 or 3;
or a pharmaceutically acceptable salt thereof.

2: The compound as claimed in claim 1 in which R¹ represents a (C₃-C₄ alkyl) sulphonyloxy group substituted by one or more fluoro.

3: The compound as claimed in claim 1 in which R¹ represents (C₃-C₄ alkoxy) group substituted by one or more fluoro.

4: The compound as claimed in claim 1 in which R² represents chloro, fluoro, cyano or methyl and n is 1 or 2.

5: The compound as claimed in claim 1 in which R⁴ represents cyclohexyl substituted by one or more of the following: hydroxy, fluoro, amino, mono or di C₁₋₃alkylamino.

6: The compound as claimed in claim 1 in which R⁴ represents piperidino substituted by one or more hydroxy.

7: The compound as claimed in claim 1 in which R⁴ represents unsubstituted piperidino but only when one of the following applies: R¹ represents 3-fluoropropylsulphonyloxy or 3,3,3-trifluoropropoxy or 3-fluoropropoxy or at least one R² represents methyl.

8: The compound as claimed in claim 1 in which R⁴ represents phenyl substituted by one or more trifluoromethoxy.

9: The compound as claimed in claim 1 in which R⁴ represents pyridyl substituted by one or more of the following: a C₁₋₄alkyl group; trifluoromethyl; or fluoro; provided that R⁴ is not 5-trifluoromethyl-2-pyridyl.

10: The compound as claimed in claim 1 in which R⁴ represents a C₄₋₇alkyl group optionally substituted by one or more hydroxy.

11: The compound as claimed in claim 1 selected from the following:
4-[2-(2,4-dichlorophenyl)-5-methyl-4-({[6-(trifluoromethyl)pyridin-3-yl]amino}carbonyl)-1H-imidazol-1-yl]phenyl 3,3,3-trifluoropropane-1-sulfonate;
4-(2-(2,4-dichlorophenyl)-5-methyl-4-{[(5-methylpyridin-2-yl)amino]carbonyl}-1H-imidazol-1-yl)phenyl 3,3,3-trifluoropropane-1-sulfonate;
4-(2-(2,4-dichlorophenyl)-4-{[(6-fluoropyridin-3-yl)amino]carbonyl}-5-methyl-1H-imidazol-1-yl)phenyl 3,3,3-trifluoropropane-1-sulfonate;
4-[2-(2,4-dichlorophenyl)-4-({[(1S,2R)-2-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[(1R,2S)-2-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[(1S,2S)-2-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[(1R,2R)-2-hydroxycyclohexyl]amino}carbonyl)-5-methyl-1H-imidazol-1-yl]phenyl 3,3,3-trifluoropropane-1-sulfonate;
4-{2-(2,4-dichlorophenyl)-5-methyl-4-[(piperidin-1-ylamino)carbonyl]-1H-imidazol-1-yl}phenyl 3-fluoropropane-1-sulfonate;
4-[2-(2,4-dichlorophenyl)-5-methyl-4-({[4-(trifluoromethoxy)phenyl]amino}carbonyl)-1H-imidazol-1-yl]phenyl 3,3,3-trifluoropropane-1-sulfonate;
3,3,3-trifluoropropane-1-sulfonic acid 4-[2-(2,4-dichlorophenyl)-4-(3-hydroxypiperidin-1-ylcarbamoyl)-5-methylimidazol-1-yl]phenyl ester;
3,3,3-trifluoropropane-1-sulfonic acid 4-[2-(2,4-dichlorophenyl)-4-(4-hydroxypiperidin-1-ylcarbamoyl)-5-methyl-imidazol-1-yl]phenyl ester;
(−)4-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate;
2-(2,4-dichlorophenyl)-5-methyl-1-[4-(3,3,3-trifluoropropoxy)phenyl]-1H-imidazole-4-carboxylic acid (2-hydroxycyclohexyl)amide;
(+)4-[2-(2,4-dichlorophenyl)-4-({[cis-2-hydroxycyclohexyl]amino}carbonyl)-5-methyl-1H-imidazol-1-yl]phenyl 3,3,3-trifluoropropane-1-sulfonate;
(+)4-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate;
(−)4-[2-(2,4-dichlorophenyl)-4-({[trans-2-hydroxycyclohexyl]amino}carbonyl)-5-methyl-1H-imidazol-1-yl]phenyl 3,3,3-trifluoropropane-1-sulfonate;
4-[4-({[(1S,2R)-2-aminocyclohexyl]amino}carbonyl)-2-(2,4-dichlorophenyl)-5-methyl-1H-imidazol-1-yl]phenyl 3,3,3-trifluoropropane-1-sulfonate;
4-[2-(2,4-dichlorophenyl)-4-({[(1S,2R)-2-(dimethylamino)cyclohexyl]amino}carbonyl)-5-methyl-1H-imidazol-1-yl]phenyl 3,3,3-trifluoropropane-1-sulfonate;
2-(2,4-dichlorophenyl)-5-methyl-N-piperidin-1-yl-1-[4-(3,3,3-trifluoropropoxy)phenyl]-1H-imidazole-4-carboxamide;
N-cyclohexyl-2-(2,4-dichlorophenyl)-5-methyl-1-[4-(3,3,3-trifluoropropoxy)phenyl]-1H-imidazole-4-carboxamide;
2-(2,4-dichlorophenyl)-N-(4,4-difluorocyclohexyl)-5-methyl-1-[4-(3,3,3-trifluoropropoxy)phenyl]-1H-imidazole-4-carboxamide;
2-(2,4-dichlorophenyl)-5-methyl-N-(5-methylpyridin-2-yl)-1-[4-(3,3,3-trifluoropropoxy)phenyl]-1H-imidazole-4-carboxamide;
2-(2,4-dichlorophenyl)-1-[4-(3-fluoropropoxy)phenyl]-5-methyl-N-piperidin-1-yl-1H-imidazole-4-carboxamide;
N-cyclohexyl-2-(2,4-dichlorophenyl)-1-[4-(3-fluoropropoxy)phenyl]-5-methyl-1H-imidazole-4-carboxamide;
2-(2,4-dichlorophenyl)-1-[4-(3-fluoropropoxy)phenyl]-N-[(cis)-2-hydroxycyclohexyl]-5-methyl-1H-imidazole-4-carboxamide;
2-(2,4-dichlorophenyl)-N-(4,4-difluorocyclohexyl)-1-[4-(3-fluoropropoxy)phenyl]-5-methyl-1H-imidazole-4-carboxamide;
2-(2,4-dichlorophenyl)-1-[4-(3-fluoropropoxy)phenyl]-5-methyl-N-(5-methylpyridin-2-yl)-1H-imidazole-4-carboxamide;
4-[2-(2,4-dichlorophenyl)-4-({[cis-3-hydroxycyclohexyl]amino}carbonyl)-5-methyl-1H-imidazol-1-yl]phenyl 3,3,3-trifluoropropane-1-sulfonate;
4-[2-(2,4-dichlorophenyl)-4-({[trans-3-hydroxycyclohexyl]amino}carbonyl)-5-methyl-1H-imidazol-1-yl]phenyl 3,3,3-trifluoropropane-1-sulfonate;
4-{2-(2-chlorophenyl)-4-[(cyclohexylamino)carbonyl]-5-methyl-1H-imidazol-1-yl}phenyl 3,3,3-trifluoropropane-1-sulfonate;
4-[2-(2-chlorophenyl)-4-({[cis-2-hydroxycyclohexyl]amino}carbonyl)-5-methyl-1H-imidazol-1-yl]phenyl 3,3,3-trifluoropropane-1-sulfonate;
2-(2,4-dichlorophenyl)-1-[4-(3-fluoropropoxy)phenyl]-5-methyl-N-piperidin-1-yl-1H-imidazole-4-carboxamide;
2-(2,4-dichlorophenyl)-N-[(cis)-2-hydroxycyclohexyl]-5-methyl-1-[4-(3,3,3-trifluoropropoxy)phenyl]-1H-imidazole-4-carboxamide;
4-{2-(2,4-dichlorophenyl)-4-[(4-hydroxycyclohexyl)carbamoyl]-5-methyl-1H-imidazol-1-yl}phenyl 3,3,3-trifluoropropane-1-sulfonate;
3,3,3-trifluoropropane-1-sulfonic acid 4-[2-(3-cyano-5-fluorophenyl)-4-(4,4-difluorocyclohexylcarbamoyl)-5-methylimidazol-1-yl]phenyl ester;
3,3,3-trifluoropropane-1-sulfonic acid 4-[2-(3-cyanophenyl)-4-(1-hydroxymethyl-3-methylbutylcarbamoyl)-5-methylimidazol-1-yl]phenyl ester;
3,3,3-trifluoropropane-1-sulfonic acid 4-[4-((2-aminocyclohexylcarbamoyl)-2-(3-cyano-5-fluorophenyl)-5-methylimidazol-1-yl]phenyl ester;
3,3,3-trifluoropropane-1-sulfonic acid 4-[2-(3-cyano-5-fluorophenyl)-4-(3-dimethylaminocyclohexylcarbamoyl)-5-methylimidazol-1-yl]phenyl ester;
3,3,3-trifluoropropane-1-sulfonic acid 4-[2-(3-cyano-5-fluorophenyl)-4-((1S,2R)-2-hydroxycyclohexylcarbamoyl)-5-methylimidazol-1-yl]phenyl ester;
3,3,3-trifluoropropane-1-sulfonic acid 4-[2-(3-cyanophenyl)-4-(2-hydroxy-cyclohexylcarbamoyl)-5-methylimidazol-1-yl]phenyl ester;
3,3,3-trifluoropropane-1-sulfonic acid 4-[2-(3-cyano-5-fluorophenyl)-4-(3-hydroxycyclohexylcarbamoyl)-5-methylimidazol-1-yl]phenyl ester;
3,3,3-trifluoropropane-1-sulfonic acid 4-[2-(2-chlorophenyl)-4-(2-hydroxy-cyclohexylcarbamoyl)-5-methylimidazol-1-yl]phenyl ester;
3,3,3-trifluoropropane-1-sulfonic acid 4-[2-(2-chlorophenyl)-4-(4,4-difluoro-cyclohexylcarbamoyl)-5-methylimidazol-1-yl]phenyl ester;
3,3,3-trifluoropropane-1-sulfonic acid 4-[2-(4-chloro-2-methylphenyl)-5-methyl-4-(piperidin-1-ylcarbamoyl)imidazol-1-yl]phenyl ester;
2-(2,4-dichlorophenyl)-5-methyl-1-[4-(3,3,3-trifluoropropoxy)phenyl]-1H-imidazole-4-carboxylic acid (3-hydroxycyclohexyl)amide;
3-Fluoropropane-1-sulfonic acid 4-[2-(2,4-dichlorophenyl)-4-((1S,2R)-2-hydroxy-cyclohexylcarbamoyl)-5-methylimidazol-1-yl]phenyl ester; and
4,4,4-trifluorobutane-1-sulfonic acid 4-[2-(3-cyano-5-fluorophenyl)-4-(1-ethyl-butylcarbamoyl)-5-methylimidazol-1-yl]phenyl ester;
and pharmaceutically acceptable salts thereof.

12. (canceled)

13: A pharmaceutical formulation comprising a compound of formula I according to claim 11 and a pharmaceutically acceptable adjuvant, diluent or carrier.

14. (canceled)

15: A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula I according to claim 1 to a patient in need thereof.

16. (canceled)

17: A process for the preparation of a compound according to claim 1 which comprises reacting a compound of formula II 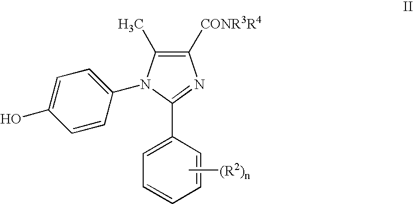
in which R², R³, R⁴ and n are as previously defined with a group R^{1A}-X in which R^{1A} represents a group such that R^{1A}O represents R¹ and X represents a leaving group at a temperature in the range of −25 to 150° C., in the presence of an inert solvent, and optionally in the presence of a base.

18: A process for the preparation of a compound according to claim 1 which comprises reacting a compound of formula III 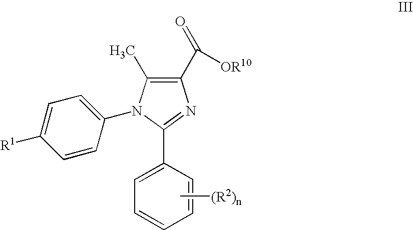
in which R¹, R² and n are as previously defined and R¹⁰ represents H or a C₁₋₆alkyl group with a compound of formula IV
R³R⁴NH₂ IV
or a salt thereof in which R³ and R⁴ are as previously defined, in an inert solvent in the presence of a Lewis Acid at a temperature in the range of −25° C. to 150° C. when R¹⁰ is a C₁₋₆alkyl group; or alternatively when R¹⁰ is H by reacting a compound of formula III with a chlorinating agent and then reacting the acid chloride produced with an amine of formula IV in an inert solvent in the presence of a base at a temperature in the range of −25° C. to 150° C.
